(19) United States
(12) Patent Application Publication
(10) Pub. No.: US 2021/0322764 A1
(43) Pub. Date: Oct. 21, 2021

(54) IMPLANTABLE COMPONENTS AND EXTERNAL DEVICES COMMUNICATING WITH SAME

(71) Applicant: Cochlear Limited, Macquarie University, NSW (AU)

(72) Inventor: Stefan Jozef MAUGER, Macquarie University (AU)

(21) Appl. No.: 17/269,025

(22) PCT Filed: Sep. 13, 2019
(86) PCT No.: PCT/IB2019/057749

§ 371 Date: Feb. 17, 2021

Related U.S. Application Data
(60) Provisional application No. 62/731,332, filed on Sep. 14, 2018.

(51) Int. Cl. A61N 1/05; H04R 25/00; A61N 1/36

(52) U.S. Cl. CPC A61N 1/0541; H04R 25/606; H04R 2225/31; A61N 1/36135; H04R 2225/67; A61N 1/36038

(57) ABSTRACT

An apparatus, including an implantable component of an implantable prosthesis, the implantable component configured to operate in at least two different operation modes, wherein a first mode is a recipient-active mode of at least 6 hours in length where data is at least sometimes streamed from the implantable component to an external component, and an alarm is applyable to the recipient via an internal alarm system of the implantable component, and a second mode is a recipient-passive mode of at least 6 hours length where the recipient sleeps, where the implantable component is powered for functional operation primarily from an external device not magnetically coupled to the recipient, where data is at least sometimes stored internally to the implantable component, and an alarm is applyable to the recipient via an internal alarm system of the implantable component.

### CROSS-REFERENCE TO RELATED APPLICATIONS

This application claims priority to U.S. Provisional Application No. 62/731,332, entitled IMPLANTABLE COMPONENTS AND EXTERNAL DEVICES COMMUNICATING WITH SAME, filed on Sep. 14, 2018, naming Stefan Jozef MAUGER of East Melbourne, Australia as an inventor, the entire contents of that application being incorporated herein by reference in its entirety.

### BACKGROUND

Hearing loss, which may be due to many different causes, is generally of two types: conductive and sensorineural. Sensorineural hearing loss is due to the absence or destruction of the hair cells in the cochlea that transduce sound signals into nerve impulses. Various hearing prostheses are commercially available to provide individuals suffering from sensorineural hearing loss with the ability to perceive sound. One example of a hearing prosthesis is a cochlear implant. Conductive hearing loss occurs when the normal mechanical pathways that provide sound to hair cells in the cochlea are impeded, for example, by damage to the ossicular chain or the ear canal. Individuals suffering from conductive hearing loss may retain some form of residual hearing because the hair cells in the cochlea may remain undamaged.

Individuals suffering from hearing loss typically receive an acoustic hearing aid. Conventional hearing aids rely on principles of air conduction to transmit acoustic signals to the cochlea. In particular, a hearing aid typically uses an arrangement positioned in the recipient's ear canal or on the outer ear to amplify a sound received by the outer ear of the recipient. This amplified sound reaches the cochlea causing motion of the perilymph and stimulation of the auditory nerve. Cases of conductive hearing loss typically are treated by means of bone conduction hearing aids. In contrast to conventional hearing aids, these devices use a mechanical actuator that is coupled to the skull bone to apply the amplified sound. In contrast to hearing aids, which rely primarily on the principles of air conduction, certain types of hearing prostheses commonly referred to as cochlear implants convert a received sound into electrical stimulation. The electrical stimulation is applied to the cochlea, which results in the perception of the received sound.

### SUMMARY

In an exemplary embodiment, there is an implantable component of an implantable prosthesis, the implantable component configured to autonomously provide a perceptible meaningful indication related to an operation of the implantable prosthesis to a recipient thereof totally via implanted componentry.

In an exemplary embodiment, there is a method, comprising powering an implanted medical device during a first temporal period where the recipient thereof is active using a body-worn external component in transcutaneous signal communication with the implanted medical device and/or using a battery implanted in the recipient and powering the implanted medical device during a second temporal period while the recipient thereof is resting using a non-body worn external component in in transcutaneous signal communication with the implanted medical device, wherein the body-worn external component is not worn during the second temporal period.

In an exemplary embodiment, there is an apparatus, comprising an implantable component of an implantable prosthesis, the implantable component configured to operate in at least two different operation modes, wherein a first mode is a recipient-active mode of at least 6 hours in length where data is at least sometimes streamed from the implantable component to an external component, and an alarm is applyable to the recipient via an internal alarm system of the implantable component wherein during the first mode, the external component is a first body worn component, and a second mode is a recipient-passive mode of at least 6 hours length where the recipient sleeps, where the implantable component is powered for functional operation primarily from an external device not worn by the recipient or from an external device different in type from the first body worn component, where data is at least sometimes stored internally to the implantable component, and an alarm is applyable to the recipient via an internal alarm system of the implantable component.

### BRIEF DESCRIPTION OF THE DRAWINGS

Embodiments are described below with reference to the attached drawings, in which:

FIG. 1 is a perspective view of an exemplary hearing prosthesis in which at least some of the teachings detailed herein are applicable;

FIG. 2 presents a functional block diagram of an example cochlear implant;

FIG. 3 illustrates an example pillow system for providing external device functionality for an implantable component.

FIG. 4 illustrates an example system that includes an implantable component and a pillow system.

FIG. 5 illustrates an example system having a separate data unit and a separate power unit.

FIG. 6 illustrates another example pillow system for providing external device functionality for an implantable component.

FIGS. 7 and 8 and 12 and 13 present schematics of some exemplary body monitoring systems;

FIGS. 9-11 present schematics of some exemplary external devices;

FIG. 14 presents an exemplary external component different in type from those of FIGS. 7, 8, 12 and 13;

FIG. 15 and FIG. 17 present exemplary implantable components;

FIG. 16 presents an exemplary magnet arrangement that is used by the device of FIG. 14 and not used by the other external components detailed herein;

FIGS. 18 and 19 provide exemplary algorithms for exemplary methods; and

FIGS. 20 and 21 provide exemplary implantable systems according to some embodiments.

### DETAILED DESCRIPTION

Embodiments are sometimes described in terms of a cochlear implant, but it is to be noted that the teachings detailed herein can be applicable to other types of hearing prostheses, and other types of sensory prostheses as well, such as, for example, retinal implants, etc. In an exemplary embodiment of a cochlear implant and an exemplary embodiment of system that utilizes a cochlear implant will first be described, where the implant and the system can be utilized to implement at least some of the teachings detailed herein.

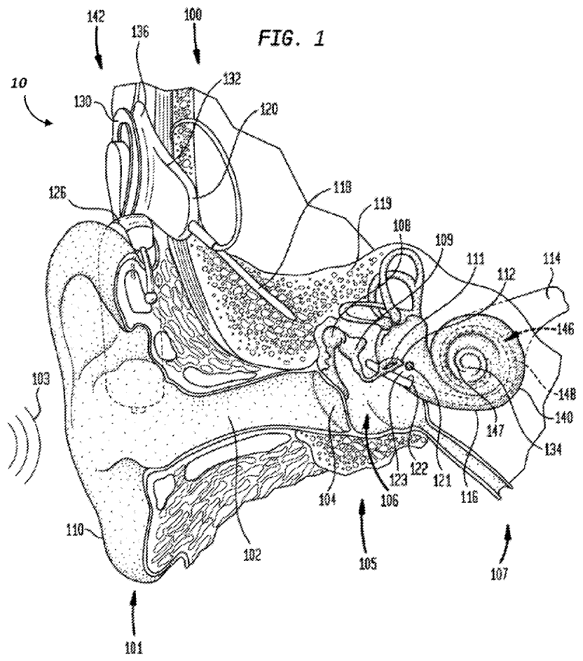

FIG. 1 is a perspective view of a cochlear implant, referred to as cochlear implant 100, implanted in a recipient, to which some embodiments detailed herein and/or variations thereof are applicable. The cochlear implant 100 is part of a system 10 that can include external components in some embodiments, as will be detailed below. Additionally, it is noted that the teachings detailed herein are also applicable to other types of hearing prostheses, such as by way of example only and not by way of limitation, bone conduction devices (percutaneous, active transcutaneous and/or passive transcutaneous), direct acoustic cochlear stimulators, middle ear implants, and conventional hearing aids, etc. Indeed, it is noted that the teachings detailed herein are also applicable to so-called multi-mode devices. In an exemplary embodiment, these multi-mode devices apply both electrical stimulation and acoustic stimulation to the recipient. In an exemplary embodiment, these multi-mode devices evoke a hearing percept via electrical hearing and bone conduction hearing.

In this regard, it is to be appreciated that the techniques presented herein may also be used with a variety of other medical devices that, while providing a wide range of therapeutic benefits to recipients, patients, or other users, may benefit from setting changes based on the location of the medical device. For example, the techniques presented herein may be used with other hearing prostheses, including acoustic hearing aids, bone conduction devices, middle ear auditory prostheses, direct acoustic stimulators, other electrically simulating auditory prostheses (e.g., auditory brain stimulators), etc. The techniques presented herein may also be used with visual prostheses (i.e., Bionic eyes), sensors, pacemakers, drug delivery systems, defibrillators, functional electrical stimulation devices, catheters, etc. Accordingly, any disclosure herein with regard to one of these types of hearing prostheses corresponds to a disclosure of another of these types of hearing prostheses or any medical device for that matter, unless otherwise specified, or unless the disclosure thereof is incompatible with a given device based on the current state of technology. Thus, the teachings detailed herein are applicable, in at least some embodiments, to partially implantable and/or totally implantable medical devices that provide a wide range of therapeutic benefits to recipients, patients, or other users, including hearing implants having an implanted microphone, auditory brain stimulators, visual prostheses (e.g., bionic eyes), sensors, etc.

In view of the above, it is to be understood that at least some embodiments detailed herein and/or variations thereof are directed towards a body-worn sensory supplement medical device (e.g., the hearing prosthesis of FIG. 1, which supplements the hearing sense, even in instances when there are no natural hearing capabilities, for example, due to degeneration of previous natural hearing capability or to the lack of any natural hearing capability, for example, from birth). It is noted that at least some exemplary embodiments of some sensory supplement medical devices are directed towards devices such as conventional hearing aids, which supplement the hearing sense in instances where some natural hearing capabilities have been retained, and visual prostheses (both those that are applicable to recipients having some natural vision capabilities and to recipients having no natural vision capabilities). Accordingly, the teachings detailed herein are applicable to any type of sensory supplement medical device to which the teachings detailed herein are enabled for use therein in a utilitarian manner. In this regard, the phrase sensory supplement medical device refers to any device that functions to provide sensation to a recipient irrespective of whether the applicable natural sense is only partially impaired or completely impaired, or indeed never existed.

The recipient has an outer ear 101, a middle ear 105, and an inner ear 107. Components of outer ear 101, middle ear 105, and inner ear 107 are described below, followed by a description of cochlear implant 100.

In a fully functional ear, outer ear 101 comprises an auricle 110 and an ear canal 102. An acoustic pressure or sound wave 103 is collected by auricle 110 and channeled into and through ear canal 102. Disposed across the distal end of ear channel 102 is a tympanic membrane 104 which vibrates in response to sound wave 103. This vibration is coupled to oval window or fenestra ovalis 112 through three bones of middle ear 105, collectively referred to as the ossicles 106 and comprising the malleus 108, the incus 109, and the stapes 111. Bones 108, 109, and 111 of middle ear 105 serve to filter and amplify sound wave 103, causing oval window 112 to articulate, or vibrate in response to vibration of tympanic membrane 104. This vibration sets up waves of fluid motion of the perilymph within cochlea 140. Such fluid motion, in turn, activates tiny hair cells (not shown) inside of cochlea 140. Activation of the hair cells causes appropriate nerve impulses to be generated and transferred through the spiral ganglion cells (not shown) and auditory nerve 114 to the brain (also not shown) where they are perceived as sound.

As shown, cochlear implant 100 comprises one or more components which are temporarily or permanently implanted in the recipient. Cochlear implant 100 is shown in FIG. 1 with an external device 142, that is part of system 10 (along with cochlear implant 100), which, as described below, is configured to provide power to the cochlear implant, where the implanted cochlear implant includes a battery that is recharged by the power provided from the external device 142.

In the illustrative arrangement of FIG. 1, external device 142 can comprise a power source (not shown) disposed in a Behind-The-Ear (BTE) unit 126. External device 142 also includes components of a transcutaneous energy transfer link, referred to as an external energy transfer assembly. The transcutaneous energy transfer link is used to transfer power and/or data to cochlear implant 100. Various types of energy transfer, such as infrared (IR), electromagnetic, capacitive and inductive transfer, may be used to transfer the power and/or data from external device 142 to cochlear implant 100. In the illustrative embodiments of FIG. 1, the external energy transfer assembly comprises an external coil 130 that forms part of an inductive radio frequency (RF) communication link. External coil 130 is typically a wire antenna coil comprised of multiple turns of electrically insulated single-strand or multi-strand platinum or gold wire. External device 142 also includes a magnet (not shown) positioned within the turns of wire of external coil 130. It should be appreciated that the external device shown in FIG. 1 is merely illustrative, and other external devices may be used with embodiments.

Cochlear implant 100 comprises an internal energy transfer assembly 132 which can be positioned in a recess of the temporal bone adjacent auricle 110 of the recipient. As detailed below, internal energy transfer assembly 132 is a component of the transcutaneous energy transfer link and receives power and/or data from external device 142. In the illustrative embodiment, the energy transfer link comprises an inductive RF link, and internal energy transfer assembly 132 comprises a primary internal coil 136. Internal coil 136 is typically a wire antenna coil comprised of multiple turns of electrically insulated single-strand or multi-strand platinum or gold wire.

Cochlear implant 100 further comprises a main implantable component 120 and an elongate electrode assembly 118. In some embodiments, internal energy transfer assembly 132 and main implantable component 120 are hermetically sealed within a biocompatible housing. In some embodiments, main implantable component 120 includes an implantable microphone assembly (not shown) and a sound processing unit (not shown) to convert the sound signals received by the implantable microphone in internal energy transfer assembly 132 to data signals. That said, in some alternative embodiments, the implantable microphone assembly can be located in a separate implantable component (e.g., that has its own housing assembly, etc.) that is in signal communication with the main implantable component 120 (e.g., via leads or the like between the separate implantable component and the main implantable component 120). In at least some embodiments, the teachings detailed herein and/or variations thereof can be utilized with any type of implantable microphone arrangement.

Main implantable component 120 further includes a stimulator unit (also not shown) which generates electrical stimulation signals based on the data signals. The electrical stimulation signals are delivered to the recipient via elongate electrode assembly 118.

Elongate electrode assembly 118 has a proximal end connected to main implantable component 120, and a distal end implanted in cochlea 140. Electrode assembly 118 extends from main implantable component 120 to cochlea 140 through mastoid bone 119. In some embodiments electrode assembly 118 may be implanted at least in basal region 116, and sometimes further. For example, electrode assembly 118 may extend towards apical end of cochlea 140, referred to as cochlea apex 134. In certain circumstances, electrode assembly 118 may be inserted into cochlea 140 via a cochleostomy 122. In other circumstances, a cochleostomy may be formed through round window 121, oval window 112, the promontory 123 or through an apical turn 147 of cochlea 140.

Electrode assembly 118 comprises a longitudinally aligned and distally extending array 146 of electrodes 148, disposed along a length thereof. As noted, a stimulator unit generates stimulation signals which are applied by electrodes 148 to cochlea 140, thereby stimulating auditory nerve 114.

Thus, as seen above, one variety of implanted devices depends on an external component to provide certain functionality and/or power. For example, the recipient of the implanted device can wear an external component that provides power and/or data (e.g., a signal representative of sound) to the implanted portion that allow the implanted device to function. In particular, the implanted device can lack a battery and can instead be totally dependent on an external power source providing continuous power for the implanted device to function. Although the external power source can continuously provide power, characteristics of the provided power need not be constant and may fluctuate. Additionally, where the implanted device is an auditory prosthesis such as a cochlear implant, the implanted device can lack its own sound input device (e.g., a microphone). It is sometimes utilitarian to remove the external component. For example, it is common for a recipient of an auditory prosthesis to remove an external portion of the prosthesis while sleeping. Doing so can result in loss of function of the implanted portion of the prosthesis, which can make it impossible for recipient to hear ambient sound. This can be less than utilitarian and can result in the recipient being unable to hear while sleeping. Loss of function would also prevent the implanted portion from responding to signals representative of streamed content (e.g., music streamed from a phone) or providing other functionality, such as providing tinnitus suppression noise.

The external component that provides power and/or data can be worn by the recipient, as detailed above. While a wearable external device is worn by a recipient, the external device is typically in very close proximity and tightly aligned with an implanted component. The wearable external device can be configured to operate in these conditions. Conversely, in some instances, an unworn device can generally be further away and less tightly aligned with the implanted component. This can create difficulties where the implanted device depends on an external device for power and data (e.g., where the implanted device lacks its own battery and microphone), and the external device can need to continuously and consistently provide power and data in order to allow for continuous and consistent functionality of the implanted device.

Technologies disclosed herein can be used to provide power and/or data to and/or retrieve data from an implantable device in situations where a recipient is not wearing an external device. The technologies can overcome one or more challenges associated therewith. In an example, disclosed technologies can provide a source of power and/or data for an implanted medical device via a system that includes a pillow or other headrest or other bodyrest component (mattress, blanket, etc.). Disclosed technologies can be configured to continuously and/or intermittently provide power and data to an implantable medical device over a period of time (e.g., substantially the entire period of time where the recipient is resting their head on the pillow). Characteristics of the continuously provided power need not be constant. For example, the power may fluctuate because the efficiency of the link between the implant and the pillow may vary as the recipient's head moves, causing the proximity of the coils to vary. The power to the implanted electronics can be smoothed for example using tank capacitors. It is common for recipients of an implanted medical device to remove their external devices while sleeping and during that time pillows are often placed in close proximity to the implanted prosthesis. In particular, auditory implants are typically disposed in close proximity to a recipients' ears and people typically place their head on a pillow such that one or both ears are close to the pillow. Thus, it can be utilitarian to incorporate a pillow into a system for providing functionality of a worn external device while a recipient of an implantable device is sleeping. For a recipient of bilateral auditory implants, it may be sufficient for night time use for only one of the two devices to function. For instance, a first device being closest to the pillow may receive sufficient power and/or data to function while a second device that is further away from the pillow may receive insufficient power and/or data to function.

Pillows and other headrests are typically significantly larger than wearable external medical devices. This allows for the components of the disclosed system to have a larger size, which can help alleviate some drawbacks caused by the system not being worn. For example, the pillow can have a relatively larger area than a typical, wearable external device. The larger area allows the pillow to have comparatively more space in which to depose a coil (or other components) for transferring power and/or data to the implanted device. For example, the area enclosed by a pillow or headrest coil can be several times larger than the corresponding area for an implant coil. A larger size coil can allow for the pillow to transmit signals over a greater distance, should the medical device not be ideally positioned relative to the pillow. By incorporating one or more aspects of an external device in relation to a pillow, functionality of the implanted device can be maintained when a recipient removes a worn external device to rest on the pillow.

With reference to an example implantable auditory prosthesis, the prosthesis can depend on an external device for both power and data. Disclosed technologies can be configured to overcome challenges associated therewith. For example, an external pillow system can include data gathering functionality (e.g., via a sound input device, such a microphone), data processing functionality (e.g., a sound processor), data transmission functionality, and/or power transmission functionality (e.g., via interleaving power and data signals sent by a coil disposed within pillow). Disclosed technologies can be useful even where the implantable auditory prosthesis is not entirely dependent on an external device for power and/or data. For example, the implantable auditory prosthesis may include a battery but disclosed technologies may nonetheless provide operational power (e.g., obviating the need for the battery to provide power and drain itself,) and/or charging power to the implantable auditory prosthesis. For instance, the implantable component may be configured to use an external power source when one is present. As another example, disclosed technologies may provide data to the implantable auditory prosthesis even where the implantable auditory prosthesis is already receiving data from another source (e.g., an implanted or external sound input device). The data (e.g., data indicative of sound) may be mixed together and used by the implanted prosthesis.

Reference may be made herein to pillows or other headrests for concision, but disclosed technologies can be can be used in conjunction with a variety of articles. Headrests can include, for example, pillows, cushions, pads, head supports, and mattresses, among others. Such articles may be covered (e.g., with a pillow case) or uncovered. Additionally, the disclosed external system components can be used with any of a variety of systems in accordance with embodiments of the technology. For example, in many embodiments, the technology is used in conjunction with a conventional cochlear implant system. FIG. 1 depicts an example cochlear implant system that can benefit from use with technology disclosed herein.

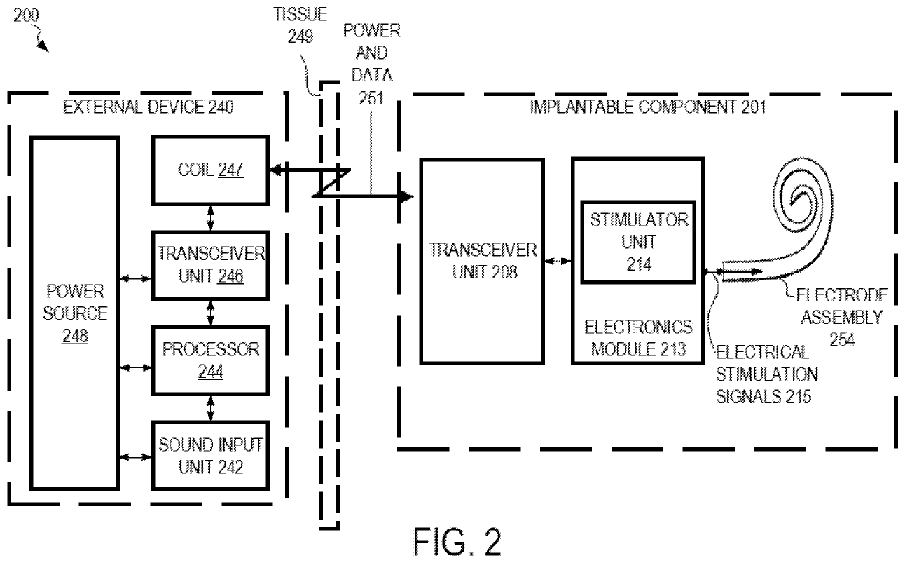

FIG. 2 is a functional block diagram of a cochlear implant system 200 that can benefit from the use of a pillow system in accordance with certain examples of the technology described herein. The cochlear implant system 200 includes an implantable component 201 (e.g., implantable component 100 of FIG. 1) configured to be implanted beneath a recipient's skin or other tissue 249, and an external device 240 (e.g., the external device 142 of FIG. 1).

The external device 240 can be configured as a wearable external device, such that the external device 240 is worn by a recipient in close proximity to the implantable component, which can enable the implantable component 201 to receive power and stimulation data from the external device 240. As described in FIG. 1, magnets can be used to facilitate an operational alignment of the external device 240 with the implantable component 201. With the external device 240 and implantable component 201 in close proximity, the transfer of power and data can be accomplished through the use of near-field electromagnetic radiation, and the components of the external device 240 can be configured for use with near-field electromagnetic radiation.

Implantable component 201 can include a transceiver unit 208, electronics module 213, which module can be a stimulator assembly of a cochlear implant, and an electrode assembly 254 (which can include an array of electrode contacts disposed on lead 118 of FIG. 1). The transceiver unit 208 is configured to transcutaneously receive power and/or data from external device 240. As used herein, transceiver unit 208 refers to any collection of one or more components which form part of a transcutaneous energy transfer system. Further, transceiver unit 208 can include or be coupled to one or more components that receive and/or transmit data or power. For example, the example includes a coil for a magnetic inductive arrangement coupled to the transceiver unit 208. Other arrangements are also possible, including an antenna for an alternative RF system, capacitive plates, or any other utilitarian arrangement. In an example, the data modulates the RF carrier or signal containing power. The transcutaneous communication link established by the transceiver unit 208 can use time interleaving of power and data on a single RF channel or band to transmit the power and data to the implantable component 201. In some examples, the processor 244 is configured to cause the transceiver unit 246 to interleave power and data signals, such as is described in U.S. Patent Application Publication Number 2009/0216296 to Meskens, which is incorporated herein by reference in its entirety for any and all purposes including for its description of techniques and devices for interleaving power and data. In this manner, the data signal is modulated with the power single, and a single coil can be used to transmit power and data to the implanted component 201. Various types of energy transfer, such as infrared (IR), electromagnetic, capacitive and inductive transfer, can be used to transfer the power and/or data from the external device 240 to the implantable component 201.

Aspects of the implantable component 201 can require a source of power to provide functionality, such as receive signals, process data, or deliver electrical stimulation. The source of power that directly powers the operation of the aspects of the implantable component 201 can be described as operational power. There are two exemplary ways that the implantable component 201 can receive operational power: a power source internal to the implantable component 201 (e.g., a battery) or a power source external to the implantable component. However, other approaches or combinations of approaches are possible. For example, the implantable component may have a battery but nonetheless receive operational power from the external component (e.g., to preserve internal battery life when the battery is sufficiently charged).

The internal power source can be a power storage element (not pictured). The power storage element can be configured for the long-term storage of power, and can include, for example, one or more rechargeable batteries. Power can be received from an external source, such as the external device 240, and stored in the power storage element for long-term use (e.g., charge a battery of the power storage element). The power storage element can then provide power to the other components of the implantable component 201 over time as needed for operation without needing an external power source. In this manner, the power from the external source may be considered charging power rather than operational power because the power from the external power source is for charging the battery (which in turn provides operational power) rather than for directly powering aspects of the implantable component 201 that require power to operate. The power storage element can be a long-term power storage element configured to be a primary power source for the implantable component 201.

In some embodiments, the implantable component 201 receives operational power from the external device 240 and the implantable component 201 does not include an internal power source (e.g., a battery)/internal power storage device. In other words, the implantable component 201 is powered solely by the external device 240 or another external device, which provides enough power to the implantable component 201 to allow the implantable component to operate (e.g., receive data signals and take an action in response). The operational power can directly power functionality of the device rather than charging a power storage element of the external device implantable component 201. In these examples, the implantable component 201 can include incidental components that can store a charge (e.g., capacitors) or small amounts of power, such as a small battery for keeping volatile memory powered or powering a clock (e.g., motherboard CMOS batteries). But such incidental components would not have enough power on their own to allow the implantable component to provide primary functionality of the implantable component 201 (e.g., receiving data signals and taking an action in response thereto, such as providing stimulation) and therefore cannot be said to provide operational power even if they are integral to the operation of the implantable component 201.

As shown, electronics module 213 includes a stimulator unit 214 (e.g., which can correspond to stimulator of FIG. 1). Electronics module 213 can also include one or more other components used to generate or control delivery of electrical stimulation signals 215 to the recipient. As described above with respect to FIG. 1, a lead (e.g., elongate lead 118 of FIG. 1) can be inserted into the recipient's cochlea. The lead can include an electrode assembly 254 configured to deliver electrical stimulation signals 215 generated by the stimulator unit 214 to the cochlea.

In the example system 200 depicted in FIG. 2, the external device 240 includes a sound input unit 242, a sound processor 244, a transceiver unit 246, a coil 247, and a power source 248. The sound input unit 242 is a unit configured to receive sound input. The sound input unit 242 can be configured as a microphone (e.g., arranged to output audio data that is representative of a surrounding sound environment), an electrical input (e.g., a receiver for a frequency modulation (FM) hearing system), and/or another component for receiving sound input. The sound input unit 242 can be or include a mixer for mixing multiple sound inputs together.

The processor 244 is a processor configured to control one or more aspects of the system 200, including converting sound signals received from sound input unit 242 into data signals and causing the transceiver unit 246 to transmit power and/or data signals. The transceiver unit 246 can be configured to send or receive power and/or data 251. For example, the transceiver unit 246 can include circuit components that send power and data (e.g., inductively) via the coil 247. The data signals from the sound processor 244 can be transmitted, using the transceiver unit 246, to the implantable component 201 for use in providing stimulation or other medical functionality.

The transceiver unit 246 can include one or more antennas or coils for transmitting the power or data signal, such as coil 247. The coil 247 can be a wire antenna coil having of multiple turns of electrically insulated single-strand or multi-strand wire. The electrical insulation of the coil 247 can be provided by a flexible silicone molding. Various types of energy transfer, such as infrared (IR), radiofrequency (RF), electromagnetic, capacitive and inductive transfer, can be used to transfer the power and/or data from external device 240 to implantable component 201.

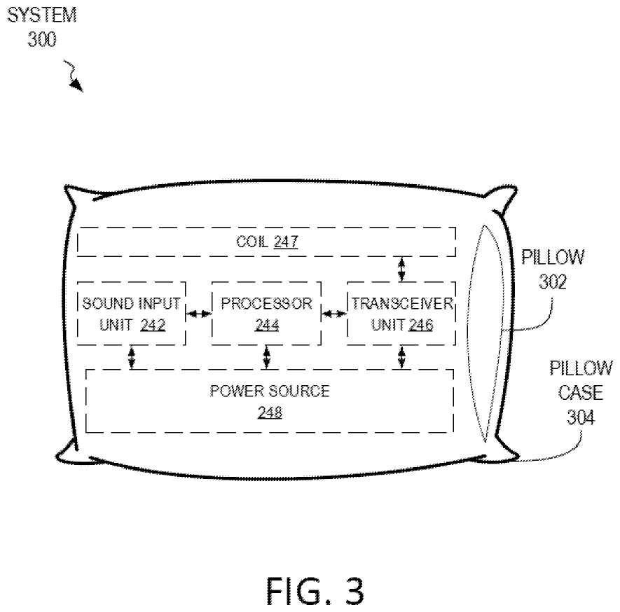

FIG. 3 illustrates an example pillow system 300 for providing external device functionality for an implantable component. The system 300 can include components similar to external device 240 of FIG. 2, which includes components for sending power and/or data signals to an implantable device. The system 300 includes a pillow or headrest 302. The pillow 302 is an article on which a person can rest, such as while sleeping. The pillow 302 can include one or more aspects to provide or increase comfort, such as being made from a soft material. Disposed within the pillow 302 can be padding material, such as foam. The pillow 302 can be partially or fully enclosed by a pillow cover 304, which can be a removable covering for the pillow 302. The cover 304 can increase the comfort of the user by, for example, including padding that inhibits the ability of the user to feel the coil 247 or another component when resting on the pillow 302.

The system 300 can include components that provide functionality and/or power for an implantable component of a medical device. The components can be disposed within or coupled to the pillow 302. These components include a sound input unit 242, a processor 244, a transceiver unit 246, a coil 247, and a power source 248. The components can be configured to be used with the pillow 302. As illustrated, the components are disposed within the pillow 302 or the cover 304 overlaying the pillow, but they need not be. One or more of the components can be disposed outside of the pillow 302 and connected to the other components via a wired or wireless connection. For example, a sound input unit 242 such as a microphone can be disposed in a stand on a bedside table and communicatively coupled to the remaining components within the pillow. In further examples, components can be disposed even more remotely from the pillow 302 (e.g., placed in another room) but can nonetheless function as part of the system 300.

In an example, the system 300 is configured to be used while a recipient of an implantable component is resting on the pillow 302 and, in particular, while resting his or her head on the pillow 302. Compared to a wearable external device, the system 300 need not be worn by a recipient, and this difference can change how the system 300 is configured. For instance, a coil of a wearable external device is often disposed in close proximity at a known orientation to an implanted device. In such a configuration, the wearable external device would likely be configured to transmit data or power using near-field electromagnetic radiation. By contrast, the coil 247 (or other transmitter) of the system 300 would often be no closer than the coil of a wearable external device, and in most cases would likely be disposed sufficiently far away as to provide power and data over some other type of transmission scheme, such as, far field electromagnetic radiation. The pillow system 300, and in particular the coil 247, can be configured to provide data and/or power using far field electromagnetic radiation. In some examples, near or far field may be used depending on a proximity detector. For instance, when a first proximity (e.g., a sufficiently short distance) to an implanted device is detected, near field electromagnetic radiation is used. When a second proximity (e.g., a sufficient far away distance) to an implanted device is detected, far field electromagnetic radiation is used.

The coil or antenna of the transceiver unit 246 can be sized or shaped to transmit or receive signals across a typical distance to an implanted device (e.g., implantable component 201) across various orientations of a recipient's head while resting on the pillow 302. For example, while typical external components for implantable medical devices are fixed (e.g., via a magnet) in a particular orientation in close proximity to the medical device, a recipient resting on the pillow 302 can be in a wider variety of orientations or configurations in relation to the coil 247. To overcome challenges associated with transmitting across this distance, the coil can be larger or otherwise configured to transmit across the wider variety of orientations than a typical, worn external device. In some examples, the coil or antenna can be integrated with a cover 304 of the pillow 302. This can allow the coil 247 to be closer to the recipient using the pillow 302 than if disposed inside the pillow 302. For example, the coil 247 can be sewn into, disposed within, attached to, coupled to, or otherwise integrated with the pillow cover 304. In some examples, the coil 247 can be positioned between the pillow 302 and the cover 304. In some examples there may be multiple coils distributed across the pillow surface with a system to select and use the coil with the best coupling to the implant.

The sound input unit 242 can have the functionality and/or configuration as described in FIG. 2 and be configured for use as part of a pillow system. In some examples, the sound input unit 242 can be disposed within the pillow 302. In these examples, the sound input unit 242 can be configured to be resistant to being muffled by the material of the pillow 302 or the recipient's head. This can involve adjusting the frequency response of the sound input unit 242. In some examples, the sound input unit 242 is disposed outside of the pillow to alleviate the sound input being muffled or picking up unwanted noise from the recipient.

The processor 244 can be as described in relation to FIG. 2 and be configured for use as a part of a pillow sound processor. In examples where the processor 244 is disposed within the pillow 302, associated structures to dissipate heat from the processor 244 can be desirable. In an example, the processor 244 can be configured to be especially low-power to reduce the amount of heat generated by the processor 244 or can be especially tolerant of high temperatures. The processor can include a large heat sink or a heat dissipation configuration suited for the purpose. In some examples, the heat sink can be integrated into one or more of the comfort features of the pillow 302, such as the filling of the pillow 302. Where the pillow 302 includes a spring, the spring can also act as a heat sink. The transceiver unit 246 can be as described in relation to FIG. 2 and be configured for use as part of a pillow sound processor. As with the processor 244, the transceiver unit 246 can be disposed within or coupled to the pillow 302. These heat dissipation strategies can also be applied to other elements such as the coil.

The power source 248 can be as described in relation to FIG. 2 and be configured for use as part of a pillow system. The power source 248 can be a power storage unit (e.g., a battery) or be components for directly receiving power from an external source, such as a wall electrical outlet. In some examples, components of the system 300 can be powered or charged wirelessly, such as via a charging pad disposed proximate the pillow 302.

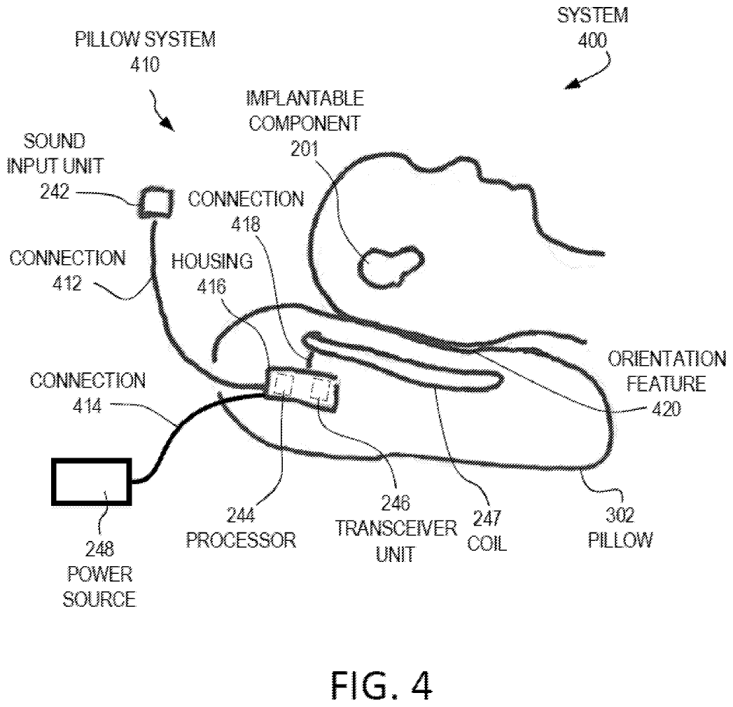

FIG. 4 illustrates an example system 400 including an implantable component 201 and a pillow system 410. The pillow system 410 includes a sound input unit 242, a processor 244, a transceiver unit 246, a coil 247, and a power source 248.

As shown, a recipient's head is resting on the pillow 302, which disposes the implantable component 201 proximate the coil 247. In this configuration, the coil 247 is able to transmit power and/or data to the implantable component. As illustrated, the recipient is not wearing a wearable external device (e.g., external device of FIG. 1). In this manner, the only power used by the implantable component 201 is from the coil 247, which makes the coil 247 the sole power source for the implantable component.

In the illustrated configuration, the sound input unit 242 is external to the pillow 302. This can facilitate placement of the sound input unit 242 in a location where it is better able to obtain sound input than within the pillow, where it can be muffled. In some examples, the sound input unit 242 can include an attachment feature (not shown) to facilitate coupling the sound input unit 242 to a particular location, such as a headboard or a wall. The sound input unit can be coupled to the processor 244 over a wired connection 412, though other configurations are also possible. For example, the sound input unit 242 can be coupled to the pillow sound processor 410 using a wireless connection.

As illustrated, the power source 248 is also external to the pillow 302 and coupled to the processor 244 through a wired connection 414. Though, again, the connection can also be made wirelessly. For example, there can be a wireless power transfer configuration, such that the power source 245 can transfer power to the components within the pillow 302 wirelessly, such as via a power coil disposed proximate the pillow 302 and a compatible power coil within the pillow and coupled to the processor 244 or a battery disposed within the pillow 302.

Where one or more of the connections 412, 414 are wired, they can connect to their respective end points (e.g., the sound input unit 242, power source 248, and housing 416) via a readily-detachable coupling, so if a recipient becomes tangled in the connections 412, 414, the connections become detached from their respective endpoints. Such a configuration can increase the recipient acceptance of the system 410.

The processor 244 and the transceiver unit 246 are illustrated as being disposed within a same housing 416. The housing 416 can be configured to be suitable for placement within a pillow 302 and can be surrounded by or include padding to increase the comfort of a recipient using the pillow 302. In some examples, the housing 416 can include an attachment feature (not shown) to facilitate anchoring the housing 416 (and thus the components within the housing) in a particular region within the pillow 302 and to resist the housing 416 from shifting positions within the pillow 302. The coil 247 is connected to the components within the housing 416 via a connection 418.

The housing 416 can also be configured for placement external to the pillow. For example, a recipient's wearable sound processor can be placed in a bedside docking station that is connected to the coil 247 and power source 248. Engagement with the docking station can automatically cause the sound processor to enter a night mode where, for example, the stimulation signal for the implant is appropriately modified (e.g., sound sensitivity is reduced) and/or the battery is recharged from the external power source 248 while the sound processor continues to operate. The docking station can also include an external sound source (e.g., a remote microphone) to supplement or replace the microphone in the wearable sound processor as needed.

As illustrated, the coil 247 is located near a location where a recipient using the pillow 302 rests his or her head. In some configurations, the pillow 302 can include an orientation feature 420 that encourages a recipient to rest his or her head on the pillow 302 in a particular orientation relative to the coil 247. For example, the orientation feature 420 can be a concavity that encourages a recipient to rest their head in a position, such that the implantable component 201 is relatively closer to the coil 247 (e.g., and thus improving a connection therebetween). Further, the pillow 302 can include an orientation feature 420 that encourages a recipient to place the pillow 302 in a particular orientation. For instance, the coil 247 can be disposed near a top portion of the pillow and the orientation feature 420 can encourage (e.g., be shaped to encourage) a top-up placement of the pillow 302, thus placing the coil 247 closer to an area where a recipient's head would rest.

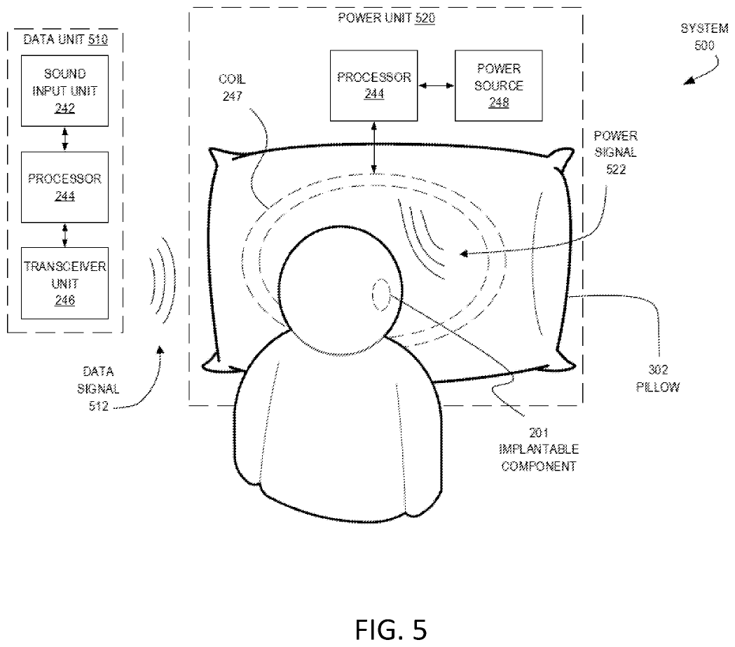

FIG. 5 illustrates an example system 500 having a data unit 510 separate from a power unit 520 (e.g., not sharing any physical components with the power unit 520). The data unit 510 is configured to send data signals 512 to the implantable component 201 and/or to receive signals from the implantable component 201, and the power unit 520 is configured to send power signals 522 to the implantable component 201.

As illustrated, the data unit 510 includes a sound input unit 242, a processor 244, a transceiver unit 246, and a power source 248. In some examples, the data unit 510 can have one or more components disposed within the pillow 302 and be configured to send a data signal 512 to the implantable component 201 using a coil 247 disposed within the pillow 302. In some examples, the data unit 510 and the power unit 520 can share the coil 247. In other examples, the data unit 510 and the power unit 520 use separate coils disposed within the pillow 302. In some examples, the transceiver unit 246 of the data unit 510 can be configured to send the data signal 512 using a wireless-communication protocol, such as BLUETOOTH (maintained by the BLUETOOTH SPECIAL INTEREST GROUP of Kirkland, Wash.). BLUETOOTH operates using radio waves having frequencies between 2.4 GHz and 2.5 GHz. In this manner, the data unit 510 can be able to communicate with the implantable component 201 across a larger distance than, for example, inductive communication. In some examples, the system 500 can concurrently transmit power and data to the implantable component 201 via distinct communication protocols. For example, the data unit 510 can use a far field protocol (e.g. BLUETOOTH) to communicate (e.g., transmit data) with the implantable component from a location remote from the pillow (e.g., a bedside table or headboard of a bed), and the power unit 520 can use a near field protocol to concurrently communicate (e.g., transmit power) with the implantable component from a location immediately adjacent the recipient's head (e.g., a coil forming part of the pillow).

While the data unit 510 can be a dedicated device, it can be advantageous to allow devices that a recipient uses on a regular basis to operate as the data unit 510. For example, a recipient's mobile phone or a recipient's wearable external medical device (e.g., external device 150) can be configured to operate as the data unit 510. For example, a phone's microphone can operate as the sound input unit 242, the phone's processor can be configured to operate as the processor 244, and a transceiver of the phone can act as the transceiver unit 246 to send a data signal 512 over BLUETOOTH (or another wireless data protocol) to the implantable component 201 based on sound received by the phone's microphone. For instance, there can be an application installed on the phone that configures the phone to operate in this manner.

In another example, a recipient can remove his or her wearable device to go to bed and place the device on a nightstand, in a charging cradle, or elsewhere. While not being worn, the wearable device still includes sound input and processing functionality, though the device can be outside of a functional range for power or data transmission. In some examples, the wearable device can still function as a data transmitter and allow the power unit 520 to take over a power functionality that would otherwise be provided by the wearable device. In some examples, the wearable device is not configured to provide data transmission when not being worn, and an adapter (not shown) can be connected to the wearable device to nonetheless allow it to provide data. For example, the adapter can receive data transmissions from the wearable device and re-transmit the data in a form more suitable for the distance to the implantable component 201.

In some examples, the data unit 510 can be located in another room from the pillow 302 to provide remote-listening functionality. In this manner, the data unit 510 can act as a baby monitor. In some examples, there can be multiple different sound input units 242, which can be placed in different locations and have their output mixed together.

The power unit 520 can be used to provide power to the implantable component 201 via coil 247 disposed in the pillow 302. As illustrated, the processor 244 and the power source 248 of power unit 520 are not disposed within the pillow 302. Instead, only the coil 247 and a connection between the processor 244 and the coil 247 are disposed within the pillow. Arranging the components in this way can increase the comfort of the pillow 302 by reducing the amount of components disposed therein.

The processors 244 and the power sources 248 of the data unit 510 and the power unit 520 can be configured to suit the respective needs of the units. For example, the processor 244 of the data unit 510 may be configured to cause the data signal 512 to be provided and the processor 244 of the power unit 520 may be configured to cause the power signal 522 to be provided by the coil. In a further example, the power unit 520 may require more power to provide its functionality than the data unit 510 does. And the respective power sources 248 may be configured accordingly. For example, the power source 248 of the power unit 520 may be a relatively large battery or a direct current converter/regulator that uses mains power. The power source 248 of the data unit 510 may be, for example, a relatively smaller battery, such as a battery that may be found in an external sound processor. In some examples, the power source 248 of the data unit 510 may nonetheless be connected to mains power for convenience or other reasons.

In some examples, the system 500 can include a hub that is physically separate from the pillow 302 and includes the data unit 510 and the power unit 520. For example, the data unit 510 and the power unit 520 can be combined in a same area or disposed in a same housing. The physically-separate hub can be remote from the pillow 302 but nonetheless electrically connected to, for example, the coil 247 via a wired or wireless connection. The hub can include a power supply for a wireless data transmitter (e.g., data unit 510) and a power supply for a wireless power transmitter (e.g., power unit 520). In some examples, the power supplies can be the same (e.g., a single power source supplies power for both) or separate.

The embodiment(s) described above with respect to FIGS. 3, 4, and 5 can enable an implanted medical device/implanted prosthesis to operate or otherwise have two or more modes of operation. By way of example only and not by way of limitation, a day mode and a night mode can be modes of operation of the medical device. It is briefly noted that the phrases day mode and night mode are not utilized herein in terms of the traditional meaning vis-à-vis the position of the sun relative to a location on the surface of the earth. Instead, these phrases are utilized herein with respect to how a human being having habits corresponding to, statistically speaking, how most people live their lives vis-à-vis day and night, where during the day, people are active, moving around, and otherwise functioning in a first manner, and during the night, people are passive, sleeping, stationary (with respect to an object, such as a bed), and otherwise functioning and a second manner vastly different from the first manner (sleeping vs. awake). In this regard, in an exemplary embodiment, a first device without an implantable battery, where such a device does not have the ability to store power for functional operation more than five (5) minutes without an external power source where without some other form of recharging), which operates in at least two modes of operation, a day mode and a night mode.

Day functional operation of an exemplary embodiment of this first device requires an external component to be worn to provide power to the implant. This external component can be similar to or otherwise the same as the external component of FIG. 1 detailed above, and thus can be a cochlear implant external device that, for example, includes a sound processor, that is in the form of, by way of example only and not by way of limitation, a behind-the-ear device (BTE device) or an off-the-ear device (OTE device), or any other external component that enables the teachings herein. The external component provides power to the implant and/or receives streamed data from the implant, and provides an external alarm. Thus, the implantable component is configured to operate only when the external component provides power to the implant, as there is no battery or other power steering device or power generating device implanted in the recipient, and is configured to stream data to the external component when so powered. In an exemplary embodiment, the implantable device is not configured to provide an alarm or otherwise provide an indication to the recipient (more on this below), at least not one that is recognizable as such by the recipient without the external component.

Still further, in an exemplary embodiment, this first device is configured to operate in a night mode, where aforementioned noted a body worn device is not in signal communication with the implantable component, and instead, the first device is configured to be placed into signal communication with the aforementioned charging pillow, which can provide power and/or data to the internal the implanted device. Thus, in an exemplary embodiment, the first component is configured to receive power and/or data from the aforementioned charging pillow. In an exemplary embodiment, data can be streamed to the charging pillow during the night mode, and/or data can be stored internally during the night mode, and upon the completion of the night mode, when the first device enters the daytime mode the data can then be streamed from the implantable component to the external device in a traditional manner. In an exemplary embodiment, the implantable component is configured to provide indications, such as an alarm, the recipient, utilizing only the implanted components, although power for such will be received from the external component.

Thus, in an exemplary embodiment there is an implantable device that operates in two different modes. The below chart provides examples of how the implantable device operates in the two modes:

| | Mode 1 (Day) | Mode 2 (Night) |
|---|---|---|
| Power | Provided from external | Provided from external device |
| | wearable device | different from that use during the |
| | | day mode. |
| Data | Streamed from the | Stored in the implanted component |
| | implanted device to the | and/or streamed from the |
| | external wearable device | implanted device to the external |
| | | device different from that used |
| | | during the day mode. |
| Alarms | Provided internally and/ | Provided internally (i.e., by the |
| | or externally (i.e., by the | implanted device). |
| | external wearable device | |
| | and/or by the implanted | |
| | device. | |

In an exemplary embodiment, during the night mode, the prosthesis system (implanted component and the external component) is configured to only provide the alarm internally. That is, the external device, such as the pillow charger, and the associated devices external to the recipient, do not and cannot provide the alarm to the recipient. In some other embodiments, the external device is also configured to provide an alarm to the recipient when operating in the second mode/night mode.

Briefly, it is noted that in at least some exemplary embodiments, the first device that has no internal battery or the like can be considered a device that cannot operate for more than X minutes without an external power source, where X is 2, 3, 4, 5, 6, 7, 8, 9, 10, 15, 20, 25, 30, 35 40, 45, 50, 55, or 60. In this regard, in at least some exemplary embodiments, there could be implanted devices that include capacitors or the like that can store power for a limited period of time. This is not to say that a device that can operate for more than five minutes without an external power source is excluded from the teachings detailed herein. This is to say that in some exemplary embodiments, the aforementioned requirements differentiate that does not have an implanted battery from a device that has an implanted battery from a device.

Thus, in an exemplary embodiment, there is a second device, that includes an internal battery. In an exemplary embodiment, such an embodiment is a device that can operate for more than y minutes without an external power source and/or without an implanted power generating device, where Y is 30, 45, 60, 90, 120, 150, 180, 240, 300, 360, 420, 480, 540, or 600. This is not to say that a device that can operate for temporal periods different than those detailed above is excluded from the teachings detailed herein vis-à-vis a device that includes an implanted battery. This is to say that in some exemplary embodiments, the aforementioned requirements differentiated a device that has an implanted battery from a device that does not have an implanted battery.

In an exemplary embodiment of the second device, the first mode of operation/day mode of operation can be such that no external component is worn or otherwise needed for operation for any of the aforementioned temporal periods. In this regard, in an exemplary embodiment, the implantable device can be considered a totally implantable device. This is not to say that the implantable device does not or otherwise cannot function with an external component. Indeed, as will be detailed below, the external component can be very utilitarian in some exemplary scenarios. During the first mode of operation/day mode of operation, power can be provided from the internal battery. Data can be stored in the implantable component while in some other embodiments, the data can also be streamed to an external device that is not body worn or otherwise that is remote from the recipient or otherwise is carried by the recipient, such as by way of example only and not by way of limitation, streamed to a personal electronics device such as a smart phone, as will be described below. In this mode, alarms or other indications are provided only internally/are provided utilizing only implanted components.

With respect to the second device, the second device is configured to operate in a second mode/night mode, where the external device, such as the pillow charger, provides power and/or data to the implantable component. In an exemplary embodiment, the implantable component is configured such that it is recharged for operation in the first mode from the pillow charger or the like. Also, in an exemplary embodiment, data can be streamed from the implanted component to the external device, such as the pillow charger. Still further, in an exemplary embodiment, the implantable component is configured to operate or otherwise function, in addition to the recharging of the battery, as a result of the power that is transcutaneously provided from the external device. In some embodiments, the device utilizes the power that is directly received from the external device, while in other embodiments, the device draws power from the battery, and thus the battery is both discharging and recharging during the second mode of operation, where the rate of discharge is less than the rate of recharge so that the battery can be recharged.

Thus, in an exemplary embodiment there is an implantable device that operates in two different modes, or in the implantable device includes a power storage device, such as a battery. The below chart provides examples of how the implantable device operates in the two modes:

| | Mode 1 (Day) | Mode 2 (Night) |
|---|---|---|
| Power | Provided from internal battery, | Provided from an external device different |
| | while in some instances, from | form any device used during mode 1, where the |
| | an external device. | power us used to recharge the internal battery |
| | | and/or power the device to functionally |
| | | operate. |
| Data | Stored internally and/or | Stored internally and/or streamed remotely, |
| | streamed remotely, such as to | such as to the pillow charger and/or to a remote |
| | a non-body worn device.* | device.* |
| Alarms | Provided internally only when | Provided internally only. |
| | no external device is present, | |
| | and provided internally and/or | |
| | externally when the external | |
| | device is present. | |

*Streaming performed through wireless Bluetooth to smartphone or external remote device, etc. Streaming can be done in real time and/or in packets. Alternatively, and/or in addition to this, communication can be intermittent in bursts of communication.

In some exemplary embodiments, there can be a third mode separate from the first and second modes. As noted above, in some embodiments, a given mode can include a streaming data feature as well as a stored data feature. In some embodiments, at least one of the aforementioned modes does not include a streaming data mode, but instead, streaming data is applied only in a third mode. In this third mode, streaming data is enabled or otherwise permitted.

Note also that in an exemplary embodiment, a third mode and/or a fourth mode can be an alarm mode, where an alarm can be raised while in one of the other modes. The user would then place an external component on the head to provide power or to stream data out from the implant. Additional details of this are described below.

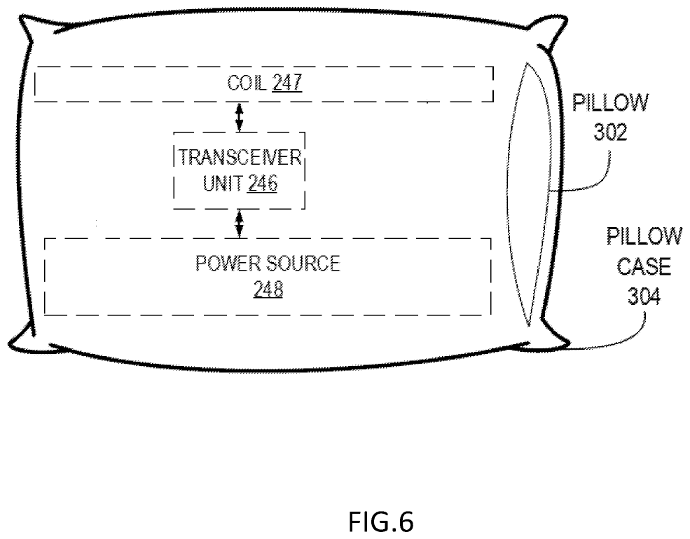

It is briefly noted that while the embodiments detailed above have generally focused on the ability of the external device to provide data or otherwise receive data from the implanted device, at least some exemplary embodiments are directed towards an external device that only powers the implantable device and/or is otherwise configured to only power the implantable device. In this regard, FIG. 6 presents such an exemplary embodiment. While FIG. 6 provides the power source and the transceiver unit located in/with the pillow, in other embodiments, consistent with the teachings detailed above, the power source and/or the transceiver unit is located away from the pillow, and can be in wired communication with the coil 274.

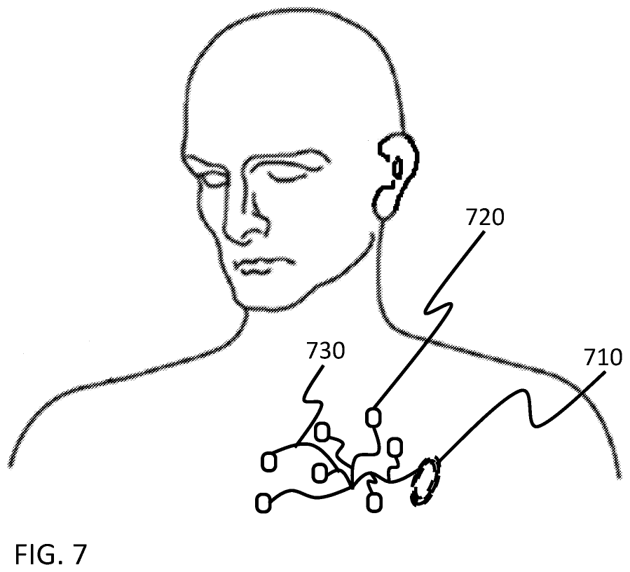

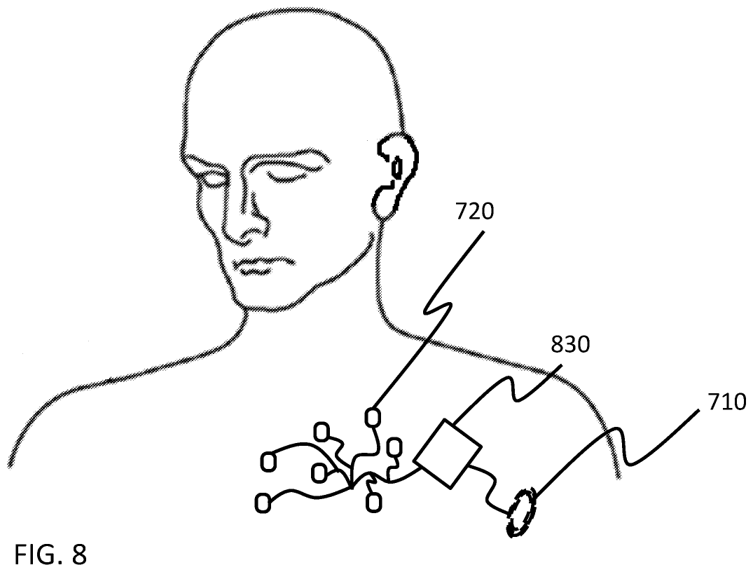

Many of the embodiments detailed above have focused on a prosthesis that is implanted in the head or otherwise includes an inductance coil that is located in the head. Indeed, the embodiments detailed above have generally focused on a hearing prosthesis, such as a cochlear implant (although it is noted that in at least some other exemplary embodiments, the hearing prosthesis is a DACI prosthesis and/or a middle ear hearing prosthesis and/or an active transcutaneous bone conduction device hearing prosthesis, all of which include an implanted radiofrequency coil such as a coil in the form of an inductance coil or any other coil that can enable the teachings detailed herein, or a radio frequency antenna or any other device that can enable communication—any disclosure herein of a cochlear implant corresponds to a disclosure in an alternate embodiment of one of the other aforementioned hearing prostheses). Some other embodiments can be embodiments that include an implanted component that is implanted elsewhere other than the head. By way of example only and not by way of limitation, in an exemplary embodiment, there can be a heart monitor and/or a heart stimulator (pacemaker), such as by way of example only and not by of limitation, the arrangement seen in FIG. 7. As seen, a heart monitor comprises a plurality of sensor/read electrodes 720, connected to an inductance coil 710 via leads 730. In this embodiment, the implanted device has no recording/storage capabilities, and requires an external device to receive a signal from the implanted inductance coil 710 so as to retrieve in real time the signal therefrom. Not shown is an implantable component that converts the electricity sensed by the sensor/read electrodes into a signal that is transmitted by the inductance coil 710. In an exemplary embodiment, the sensor arrangement seen in FIG. 7 is an implanted EKG sensor arrangement. FIG. 8 depicts another arrangement of an implantable sensor arrangement that again includes the sensor/read electrodes 720 and the leads 730. Here, in this embodiment, there is a housing 830 which includes circuitry that is configured to receive the signals from the leads from the electrodes 720 and record the data therefrom or otherwise store the data, and permits the data to be periodically read from an external device when the external device comes into signal communication with the implanted inductance coil 710. Alternatively, and/or in addition to this, the circuitry is configured to periodically energize the inductance coil 710 so as to provide the data to the coil 710 so that it creates an inductance signal which in turn communicates with an external component that reads the signal and thus reads the data associated with the electrodes. Thus, in at least some exemplary embodiments, the implantable apparatus is configured to stream the data. Still further, in some embodiments, the data is not streamed, but instead provided in bursts.

Any arrangement that can enable the data associated with the read electrodes to be provided from inside the recipient to outside the recipient can be utilized in at least some exemplary embodiments. In this regard, traditional implanted EKG sensor arrangements can be obtained and modified so as to implement the teachings detailed herein and/or variations thereof.

It is noted that some embodiments of the sensor arrangement of FIG. 8 includes an implanted battery or otherwise implanted power storage arrangement, while in other embodiments the arrangement specifically does not, making the arrangement akin to the embodiment of FIG. 7.

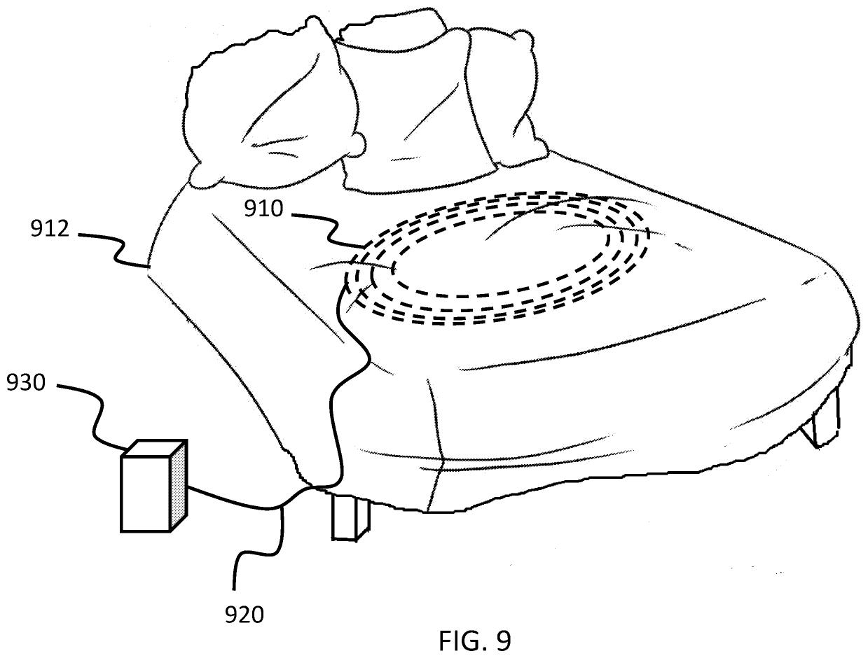

FIG. 9 presents an alternate embodiment of an external device configured to communicate with an implantable component. Here, the inductance coil 910 is associated with a bed 912, as can be seen. In an exemplary embodiment, the coil 910 can be embedded (no pun intended) into a mattress of the bed and/or can be located between the mattress of the bed, on top of the mattress, and the covering sheet upon which a human typically lays. In an exemplary embodiment, the coil can be embedded in the covering sheet that lays over the mattress. In an exemplary embodiment, the coil can be located in an outer sheet of the bed, and thus when the recipient is sleeping or otherwise lying in bed, the coil 910 can be located above/over the person. In a similar vein, the coil 910 can be located in between two or more covering sheets. Still further, in an exemplary embodiment, a plurality of coils can be utilized. One or more of the coils can be located below with the person while sleeping, and another coil can be located above the person while sleeping this can have utilitarian value with respect to always maintaining a coil to the implanted component irrespective of whether the recipient is sleeping on his or her back or on his or her stomach.

In an exemplary embodiment, the apparatus of FIG. 9 has the functionality of any of the pillows detailed above, except that the coil is associated with the bed instead of the pillow as just described. As seen, the coil number 910 is connected to a black box 930 via lead 920. In an exemplary embodiment, black box 930 is a housing that contains electronic components of the like, such as any of the components detailed above with respect to the pillow charger, and thus by way of example, can include a transponder and/or a power source, etc. logic and control circuitry, such as a programmed microprocessor or the like can be contained in the housing. Indeed, in an exemplary embodiment, the black box 930 can be a personal computer or the like, in the lead 920 can be a USB cable. It is noted that in an exemplary embodiment, black box 930 can be configured to be plugged into household electricity or the like. Black box 930 can also include Wi-Fi and/or Bluetooth technology components, such as a transmitter and/or a receiver, to communicate with a household Wi-Fi system (or a hotel Wi-Fi system), for example.

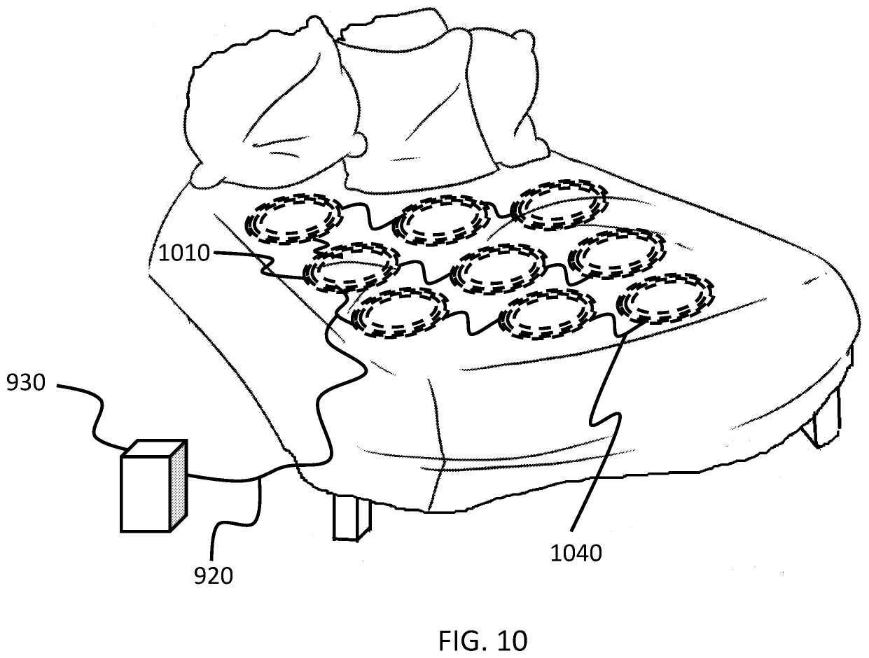

FIG. 10 presents an alternate embodiment of the embodiment of FIG. 9, where instead of a large coil, a plurality of small coils is utilized, as can be seen. More specifically, the embodiment shown in FIG. 10 includes nine separate RF inductance coil's 1010, connected to each other or otherwise connected to the black box 930 via leads 1040 in combination with lead 920. The coils can be arrayed in a manner concomitant with the coil(s) of the embodiment of FIG. 9. It is also noted that while the embodiment of FIG. 10 depicts nine coils, fewer coils, or more coils can be utilized. Any arrangement that can enable the teachings detailed herein can be utilized in at least some exemplary embodiments.

The embodiment of FIG. 9 and/or FIG. 10 can enable signal communication with the implant located outside the head of the recipient, such as the implants of FIG. 7 and FIG. 8. Such can be enabled in a manner analogous to the teachings associated with the pillow charger detailed above.

It is also noted that the embodiment of FIG. 9 and/or FIG. 10 can be utilized in combination with a pillow charger. Indeed, in an exemplary embodiment, a modified pillow can be utilized where the recipient hugs or otherwise lies against the pillow when he or she is sleeping on his or her side, if such person is such a sleeper.

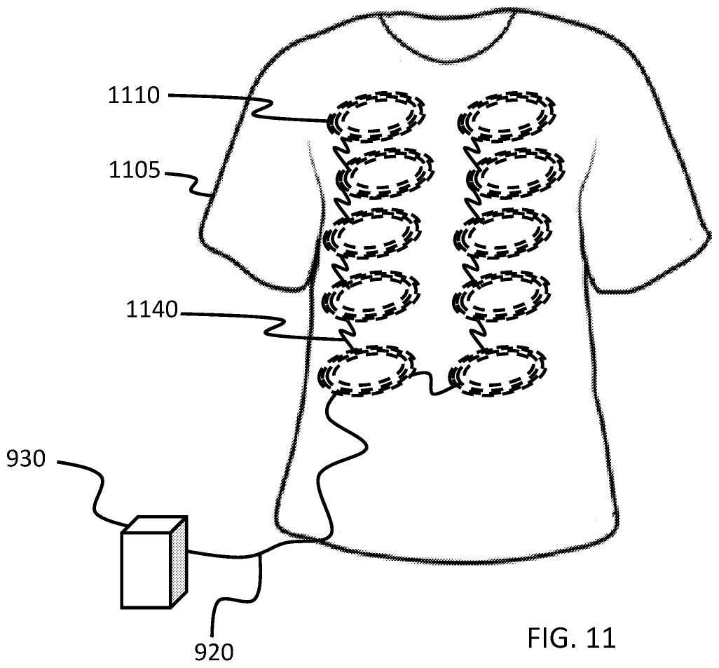

FIG. 11 depicts an alternate embodiment of an external device in the form of a tunic or t-shirt or blouse, etc., in which is located or otherwise has connected thereto a plurality of coils 1110 which are in wired communication via leads 1140 and 920 with the black box 930. In an exemplary embodiment, the coils can be located in the front, and/or on the back of the tunic. In some embodiments, the coils can be located on the side of the tunic. Such can have utilitarian value with respect to communicating with a device that works in combination with a kidney prosthesis by way of example. In an exemplary embodiment, the recipient sleeps or otherwise rests while wearing the tunic. The embodiment of FIG. 11 can have any of the features associated with the charging pillow detailed above.

It is noted that the embodiment of FIG. 11 is designed to be a stationary embodiment in that the recipient is not going to be moving around while wearing the tunic. Indeed, in an exemplary embodiment, the tunic is restricted to scenarios of use where the recipient is lying in bed. In this regard, in an exemplary embodiment, black box 930 is configured to be stationary and otherwise requires household power (110 VAC, 220 VAC, 50-60 Hz, etc.) to operate. It is noted that in at least some embodiments, an AC to DC adapter and/or a voltage drop device and/or electrical isolation device is located in the box 930 or remote from the box 930 so as to reduce the possibility however unlikely of there being a path for one 110 and/or 220 VAC reaching the recipient. In an exemplary embodiment, box 930 is powered in a manner akin to how a laptop computer is powered, where the inverter/voltage drop box is located remote from the computer.

Thus, the tunic of FIG. 11 is a design that is meant for use during the aforementioned nighttime mode and specifically not provided for use during the aforementioned daytime mode. That said, in at least some exemplary embodiments, in a variation of the embodiment of FIG. 9 or FIG. 10, a “sitting quilt” can have the aforementioned features associated with these embodiments. This can be used when a person is laying on a couch or the like, while not necessarily sleeping, but in a position where he or she is not going to move very much for extended periods of time.

Indeed, in an exemplary embodiment, the features associated with the embodiment 9 and/or embodiment 10 can be combined with a heating blanket or a cooling blanket. Thus, in an exemplary embodiment, not only can the blankets have the signal communication functionalities detailed herein, but can also provide for thermal transfer or the like.

To be clear, the embodiments of FIGS. 9, 10, and 11 are designed to provide relatively large amounts of misalignment between the implanted component and the external coils. In many respects, these devices are inefficient relative to a traditional body worn external device, such as where the external device has a coil that is aligned with the implantable coil via a magnet, such as is the case with a cochlear implant. By way of example only and not by way of limitation, on a power consumption basis, before accomplishing the exact same functions, the devices of the embodiments of FIGS. 9, 10, and 11 are at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 or more times less efficient than the body worn devices having the coil alignments detailed herein all other things being equal. This can also be the case with respect to the above detailed pillow charger.

It is also noted that in an exemplary embodiment, the amount of data that can be transferred from the external component to the implanted component for a given amount of power for a given amount of time, all other things being equal, is at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 or more times lower than the body worn device is having the coil alignments detailed herein, all other things being equal. Again, this can also be the case with respect to the above detailed pillow charger.

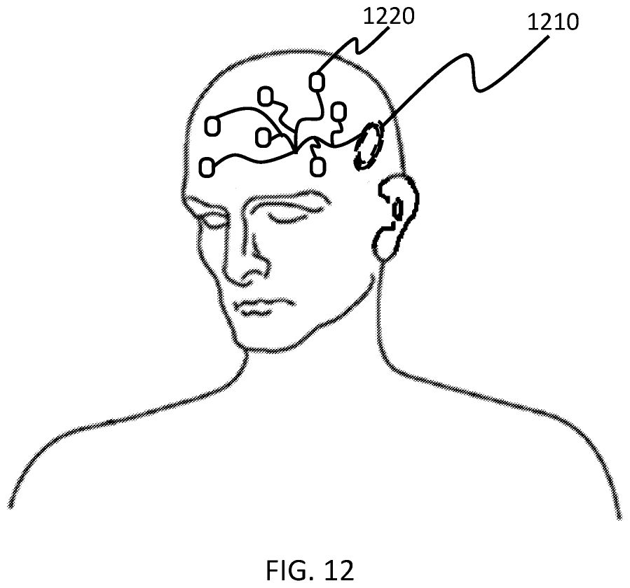

FIG. 12 provides an exemplary embodiment of an EEG system that is implanted in the recipient, where read/sense electrodes 1220 are arrayed inside a recipient's head and in signal communication with a coil 1210 via electrical leads. In this embodiment, the implanted device has no recording/storage capabilities, and requires an external device to receive a signal from the implanted inductance coil 1010 so as to retrieve in real time the signal therefrom. Not shown is an implantable component that converts the electricity sensed by the sensor/read electrodes into a signal that is transmitted by the inductance coil 710. In an exemplary embodiment, the sensor arrangement seen in FIG. 10 is an implanted EEG sensor arrangement.

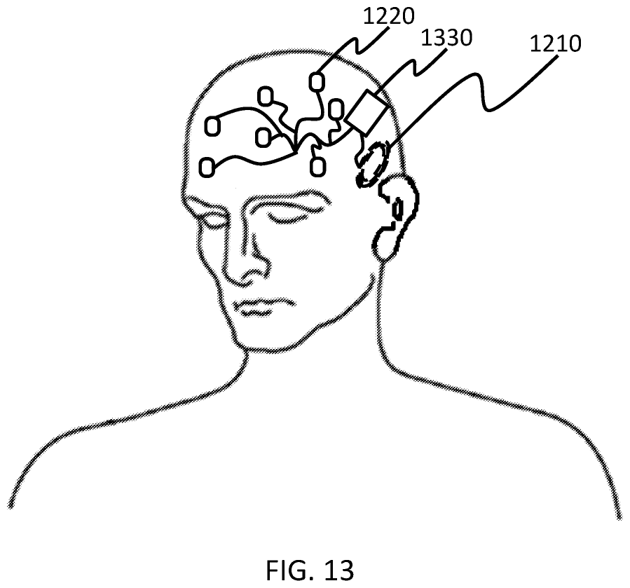

FIG. 13 depicts another arrangement of an implantable sensor arrangement that again includes the sensor/read electrodes 1220 and the leads. Here, in this embodiment, there is a housing 1330 which includes circuitry that is configured to receive the signals from the leads from the electrodes 1220 and record the data therefrom or otherwise store the data, and permits the data to be periodically read from an external device when the external device comes into signal communication with the implanted inductance coil 1210. Alternatively, and/or in addition to this, the circuitry is configured to periodically energize the inductance coil 1210 so as to provide the data to the coil 1210 so that it creates an inductance signal which in turn communicates with an external component that reads the signal and thus reads the data associated with the electrodes. Thus, in at least some exemplary embodiments, the implantable apparatus is configured to stream the data. Still further, in some embodiments, the data is not streamed, but instead provided in bursts.

Any arrangement that can enable the data associated with the read electrodes to be provided from inside the recipient to outside the recipient can be utilized in at least some exemplary embodiments. In this regard, traditional implanted EEG sensor arrangements can be obtained and modified so as to implement the teachings detailed herein and/or variations thereof.

It is noted that some embodiments of the sensor arrangement of FIG. 13 includes an implanted battery or otherwise implanted power storage arrangement, while in other embodiments the arrangement specifically does not, making the arrangement akin to the embodiment of FIG. 12.

It is also noted that in at least some exemplary embodiments, the embodiments of FIGS. 9 and 10 can be utilized with other types of prostheses than those detailed herein. By way of example only and not by way of limitation, a penile implant can be powered utilizing the embodiment of FIG. 9 or FIG. 10, where the inductance coil that powers the implant can be located in the mid-body sections of the recipient. In an exemplary embodiment, power can be transcutaneously transmitted from the coil to the implanted prosthesis in a manner such that the implanted prosthesis can be utilized without the recipient having to wear anything (nothing at all is worn on the recipient. Indeed, this is traditional garb in many cultures when performing acts where such an implant can have utilitarian value. Thus, the teachings detailed herein can be utilitarian with respect to supporting cultural aspects associated with a given prosthesis.

Indeed, in an exemplary embodiment, there are such implantable medical devices in the form of bladder valves and bladder pumps. Any of the teachings detailed herein can be utilized with such components. By way of example only and not by way of limitation, in an exemplary embodiment, the coils of the embodiment of FIG. 9 and FIG. 10 can be utilized to power the implanted bladder valves. Devices that relieve pressure on the prostate or the like can also be powered by the coils according to some of the embodiments.

In view of the above, it is to be understood that in at least some exemplary embodiments, there are traditional implanted EEG and EKG sensor systems that are configured to communicate with the external devices detailed herein. In an exemplary embodiment, the structure implanted in the recipient is the exact same thing as these traditional sensor systems, with the exception that they have been modified to operate in the various modes detailed herein, such as by way of programming or by structural modification or by the inclusion of logic circuitry, etc.

In an exemplary embodiment, the sensory systems of FIGS. 12 and 13 are used in combination with the pillow charger detailed above for communication and/or powering and/or charging. Any disclosure herein of the use of the pillow charger associated with the hearing prosthesis detailed above also corresponds to the use of the pillow charger for data transfer and/or for powering and/or charging the sensor systems of FIGS. 12 and 13 or any other sensor systems detailed herein, just as any disclosure associated with the pillow charger vis-à-vis the cochlear implant also corresponds to a disclosure of such with respect to an implanted middle ear prosthesis, a DACI and an active transcutaneous bone conduction device. Note also that any disclosure herein of use of the pillow charger or any other external component corresponds to a disclosure of use with a so-called retinal implant or bionic eye. Thus, in an exemplary embodiment, the implantable component is any of the aforementioned systems.

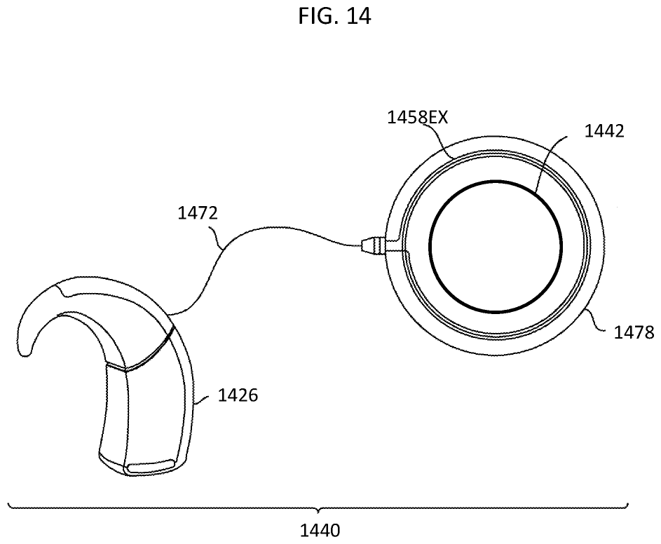

It is noted that while the embodiments detailed herein are described in terms of utilizing an external device that is fixed or otherwise relatively immobile to communicate and/or power the implanted component, it is to be understood that these devices can also be powered by their traditional external components. In this regard, FIG. 14 depicts an exemplary external component 1440. External component 1440 can correspond to external component 142 of the system 10. As can be seen, external component 1440 includes a behind-the-ear (BTE) device 1426 which is connected via cable 1472 to an exemplary headpiece 1478 including an external inductance coil 1458EX, corresponding to the external coil of FIG. 1. As illustrated, the external component 1440 comprises the headpiece 1478 that includes the coil 1458EX and a magnet 1442. This magnet 1442 interacts with the implanted magnet (or implanted magnetic material) of the implantable component to hold the headpiece 1478 against the skin of the recipient. In an exemplary embodiment, the external component 1440 is configured to transmit and/or receive magnetic data and/or transmit power transcutaneously via coil 1458EX to the implantable component, which includes an inductance coil. The coil 1458X is electrically coupled to BTE device 1426 via cable 1472. BTE device 1426 may include, for example, at least some of the components of the external devices/components described herein.

Accordingly, in an exemplary embodiment, external component 1440 can be utilized with the implantable component that is an implantable hearing prosthesis and/or an implantable retinal implant and/or an implantable sense prosthesis as detailed herein where the implanted coil is implanted near or in the head. In this regard, the external device of FIG. 14 can be utilized in combination with the exemplary EEG system of FIGS. 12 and 13. Indeed, in an exemplary embodiment where, for example, the implanted coil of the EKG system detailed herein is located in the upper reaches of the torso, such as at the top of the chest, it is possible to utilize the external device 1440 with such a system by snaking the lead 1472 downward through a person's shirt collar or the like to the person's chest or shoulder. That said, in alternate embodiments, a specialized external device especially for the EKG system can be utilized, where, for example, the non-coil portions (e.g., the equivalent of the BTE component 1426) is worn on a chain around the person's neck like a pendant, and the coil is magnetically adhered to the coil inside the person. Further, an off-the-ear (OTE) device could be used, which can be a single unit located over the coil, wherever such is located. This device would not be on a pendant, but instead could be held by a magnet, etc., to the recipient.

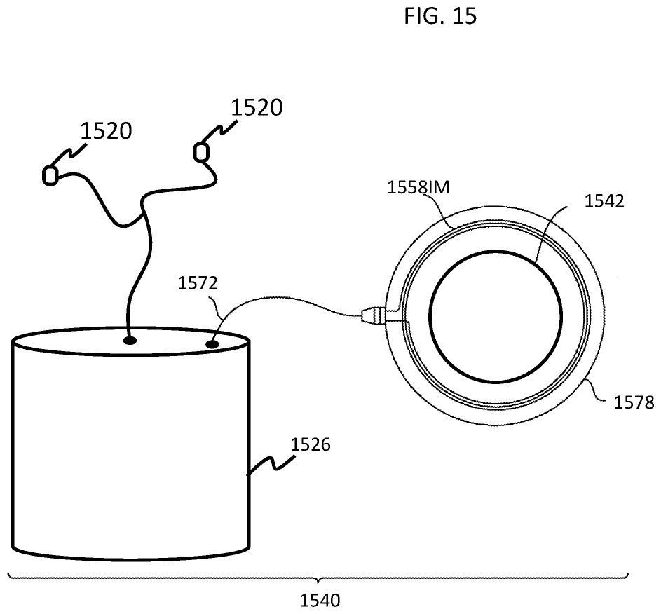

With respect to the implantable device, FIG. 15 provides an exemplary functional arrangement of an implantable device 1540 that is configured to transcutaneously communicate via an inductance field with the external device of FIG. 14 or an analogous device. Implantable component 1540 can correspond to the implantable component of the system 10 of FIG. 1. Alternatively, and/or in addition to this, the implantable component of FIG. 15 can correspond by way of representation to the implantable component of the EEG embodiment or the EKG embodiment or the retinal implant embodiment. As can be seen, external component 1540 includes an implantable housing 1526 which is connected via cable 1572 to an exemplary implanted coil apparatus 1578 including an implanted inductance coil 1558IM, corresponding to the external coil of FIG. 1 in this exemplary embodiment, where FIG. 15 represents the cochlear implant of FIG. 1. As illustrated, the implantable component 1540 comprises an implanted inductance communication assembly that includes the coil 1558IM and a magnet 1542. This magnet 1152 interacts with the external magnet of the implantable component to hold the headpiece 1478 against the skin of the recipient. In an exemplary embodiment, the implantable component 1540 is configured to transmit and/or receive magnetic data and/or receive power transcutaneously via coil 1558IM from the external component, which includes an inductance coil as detailed above. The coil 1558IM is electrically coupled to the housing 1526 via cable 1572. The housing 1526 may include may include, for example, at least some of the components of the implantable component of FIG. 1, such as for example, the stimulator of the cochlear implant where the embodiment of FIG. 15 represents such.

Implantable component 1540 also includes a stimulating assembly which includes leads extending from the housing 1526 that ultimately extend to electrodes 1520, as seen. In the embodiment where FIG. 15 represents the implantable component of the cochlear implant, electrodes 1520 and the associated leads functionally represents the electrode assembly of a cochlear implant, although it is specifically noted that in a real cochlear implant, electrodes 1520 would be supported by a carrier member instead of being “free” as shown. That said, in an exemplary embodiment, FIG. 15 can represent the EEG and/or the EKG systems detailed above, where the electrodes 1520 are read/sense electrodes. Still further, in an exemplary embodiment, the implantable component of FIG. 15 can represent the retinal implant. Note further, that in an exemplary embodiment, the electrodes 1520 are replaced with mechanical actuators, and thus the embodiment of FIG. 15 represents an active transcutaneous bone conduction device and/or a middle ear implant, etc.

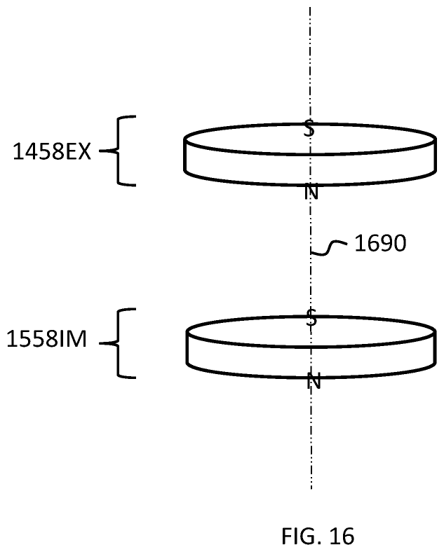

In this regard, FIG. 15 is presented for conceptual purposes to represent how the external component of FIG. 14 communicates with the implanted component. Along these lines, in an exemplary embodiment, the external component's magnet magnetically aligns with the implantable component's magnet, thus aligning the external coil with the implanted coil. This can have utilitarian value as aligning the coils provide efficiency relative to that which would be the case if the coils are misaligned. By way of example only and not by way of limitation, in an exemplary embodiment, the magnets are disk magnets having the north-south polarity aligned with the axis of rotation of the disks. In this regard, the magnets want to align the magnetic fields with one another, and thus by holding the respective coils at predetermined and control distances from the respective magnets utilizing the structure of the external component and/or the implantable components (e.g., a silicone body) the coils will become aligned with each other because the magnets will become aligned with each other. FIG. 16 depicts how the respective magnets aligned with one another with respect to their north south poles. As can be seen, both magnets aligned about axis 1690. This has the effect of aligning the respective coils.

Accordingly, in an exemplary embodiment, implantable component 1540 can be utilized with the external component that is an external component of a hearing prosthesis and/or an external component of a retinal implant and/or an external component of a sense prostheses as detailed herein. In this regard, the implantable device of FIG. 15 can represent the exemplary EEG system of FIGS. 12 and 13.

Accordingly, embodiments include utilizing an external component to transcutaneously communicate with an implantable component utilizing inductance field technology to transfer power and/or data and/or receive data. In an exemplary embodiment, the external component and the implantable component include magnets such that the respective inductance coils are relatively aligned. Embodiments also include utilizing an external component to transcutaneously communicate with the implantable component utilizing inductance field technology to transfer power and/or data and/or receive data, but in these embodiments, the external component specifically does not include magnets and/or the utilization of the external component is utilized such that the respective inductance coils are not aligned in the relative manner that would be the case utilizing the magnet arrangements that are present in the embodiments of FIG. 14 and FIG. 15. That is, in an exemplary embodiment, the external device does not include the external magnet 1458EX and/or to the extent the external device includes a magnet, the magnet is not utilized to align the coils with the implanted coil(s).

In view of the above, the embodiments such as the pillow charger and/or the accoutrements of the bed embodiments detailed above can provide for long term EEG monitors and/or long term ECKG monitors, etc. the teachings detailed herein can enable an EEG and/or EKG monitor system which has multiple modes of operation. Further, the teachings detailed herein can enable the use of a remote power source, and/or a remote data streaming capability.

The teachings detailed herein can enable implantable devices that have specific modes of operation depending on the user's activity or use of the device. For instance, as detailed above, by way of example only and not by way of limitation, day and night modes with separate characteristics. Further as seen above, there can be at least two device arrangements where multiple modes of operation can be utilitarian. A simple device contains no internal power supply (battery) and requires power to be transferred from an external device to operate. Without the power transfer, the device cannot operate or otherwise function. In an exemplary embodiment, the EKG and/or EEG system can be analogous to a cochlear implant system without an implantable battery. On an opposite side of the spectrum is a more complex device that includes an internal battery and can operate with no power from an external device, where this implantable device is configured to operate for at least 2, 3, 4, 5 6, 7, 8, 9, or 10 or more hours without the external device providing power to the implantable device. This is analogous in some respects to a totally implantable cochlear implant that includes an implantable battery/power source.

In some embodiments, an implantable EEG monitor/EKG monitor with multiple devices would have different operating regimes for power, data, and alarms depending on its mode of operation.

Some embodiments enable an implantable EEG monitor and/or an implantable EKG monitor to monitor EEG/EKG continuously, day or night. Part of this process can be to stream EKG/EEG data from the implant to an external component. The teachings detailed herein can enable an implantable EEG/EKG monitor and/or any other type of monitor to have distinct operational regimes for day and night operation. In this regard, EEG/EKG monitoring is typically performed with electrodes placed on the head/skin/torso/etc. Due to user movement, these electrodes often need reattachment, limiting the practical recording duration to around 1 week. The teachings detailed herein enable an implantable EEG/EKG monitoring device which avoids the need to reattach these electrodes, enabling the practical recording duration to well beyond one week, such as 2, 3, 4, 5, 6, 7, 8, 9, 10, 11, 12, 13, 14, 15, 16, 17, 18, 19, or 20 weeks or months or years or decades.

In view of the above, an exemplary embodiment includes an apparatus, comprising an implantable component of an implantable prosthesis, the implantable component configured to operate in at least two different operation modes. In an exemplary embodiment, the implantable component is an implantable component of the EKG monitoring device and/or an EEG monitoring device, in other embodiments, the implantable component can be a sense prosthesis or a tissue stimulating prosthesis, such as a pacemaker, etc. In an exemplary embodiment, a first mode is a recipient-active mode of at least A hours in length where data is at least sometimes streamed from the implantable component to an external component, and an alarm is applyable to the recipient via an internal alarm system of the implantable component. In an exemplary embodiment, A is at least 1, 2, 3, 4, 5, 6, 7, 8, 9, 10, 11, 12, 13, 14, 15, 16, 17, or 18 hours long. In an exemplary embodiment, a second mode is a recipient-passive mode of at least B hours length where the recipient sleeps, where the implantable component is powered for functional operation primarily from an external device not magnetically coupled to the recipient, where data is at least sometimes stored internally to the implantable component, and an alarm is applyable to the recipient via an internal alarm system of the implantable component. In an exemplary embodiment, B is at least 1, 2, 3, 4, 5, 6, 7, 8, 9, 10, 11, 12, 13, 14, 15, 16, 17, or 18 hours long.

By way of example only and not by way of limitation, data can be stored in an onboard internal memory of the implantable component. Still further by way of example only and not by way of limitation, data can be streamed via the implanted inductance coil to an external inductance coil. With respect to the first mode, in at least some exemplary embodiments, concomitant with the scenarios of use that utilize a dedicated body worn external device that is configured with an external magnet, the data that is streamed via the implanted inductance coil during the first mode of operation is streamed to a portion of the external component where the external magnet is utilized to hold a portion of the external device to the skin of the recipient and also align the external inductance coil to the internal coils. Still further in this embodiment, the external device can be utilized to provide power to the implantable device.

With respect to the alarm feature, in an exemplary embodiment, the alarm that is appliable to the recipient via an internal alarm system of the implantable component can be an arrangement where the implantable component is configured to provide stimulation to tissue of the recipient that evokes a sensory percept in predetermined manner that is noticeable by the recipient and has meaning to the recipient. Some additional details of this will be described below, but in an exemplary embodiment, this could be the utilization of an implanted electrode analogous to an electrode of a cochlear implant to evoke a hearing percept indicative of an alarm. Note that this may not necessarily be spoken words or the like, but instead can be a more generalized sound having a pattern that the recipient recognizes as being alarm or otherwise providing data to the recipient. More details of this will be described below.

It is noted that in an exemplary embodiment of the above exemplary embodiment, the implantable component is powered for functional operation during the second mode of operation primarily from an external device that is not worn by the recipient and/or by an external device that is different in type from the body worn component utilized during the first mode of operation. A non-body worn external device can correspond to the pillow charger and/or the sheet chargers detailed above, whereas an external device that is different type from the body worn component utilized during the first mode of operation can correspond to the shirt embodiment detailed above, which is different than the external device associated with FIG. 14 and those analogous thereto. In many respects, this is concomitant with the embodiments detailed above with the external device does not have a magnet or otherwise utilize a magnet to align the external coil with the implantable coil. This as contrasted to the external device utilized during the first mode of operation with the external device has a magnet and that magnet is utilized to align the external coil of the implantable coil.

In an exemplary embodiment, the first mode is such that an alarm is also applyable to the recipient via an external alarm of an external component in signal communication with the implantable component. In this regard, the alarm can be an alarm on the external component, such as the BTE device. This can be a flashing light or can be an audible alarm or a tactile alarm, etc. Note also that the phrase “alarm” includes any data that is provided to the recipient that is interpreted by the recipient as an alarm or otherwise an indication that an action should be taken or that an event is going to occur that can have a deleterious effect on the recipient or associated with the recipient. By way of example only and not by way of limitation, the alarm could be a low battery alarm with respect to embodiments that include an implanted power source. The alarm could be a voice annunciator stating the words “implanted battery has a low charge,” or can be a series of beeps or noises, where the pattern is predetermined and the recipient knows what the pattern means or otherwise can figure out what pattern means in short order (e.g., a long beep followed by a short beep followed by a long beep can be indicative of a low battery alarm). Again, additional features of the alarm will be described in greater detail below.

In an exemplary embodiment, the first mode is such that an alarm is only applied internally. That is, in an exemplary embodiment, the external device is not configured to provide any kind of alarm to the recipient. In this exemplary embodiment, the implantable system is a system that relies totally on an implantable component to provide the alarm.

In an exemplary embodiment, the first mode is such that the data is always streamed from the implantable component to the external component. In an exemplary embodiment, the data is never stored in the implantable component, at least during the first mode. In an exemplary embodiment, the data is never stored in the first mode or in the second mode. That said, in an alternate embodiment, the data can be stored in the first mode. Thus, in an exemplary embodiment, the first mode is such that the data is at least sometimes stored internally in the implantable component. Further, the second mode can be such that the data is at least sometimes stored internally in the implantable component as well. Also, in an exemplary embodiment, the second mode is such that the data is at least sometimes streamed from the implantable component to an external component. In an exemplary embodiment, the external component can be any of the “fixed” external components detailed herein.

Further, in an exemplary embodiment, there is an apparatus, comprising an implantable component of an implantable prosthesis, the implantable component configured to operate in at least one operation mode, including a first mode is a recipient-passive mode of at least 4 hours length where the recipient sleeps, where the implantable component is powered for functional operation primarily from an external device not magnetically coupled to the recipient, where data is at least sometimes stored internally to the implantable component, and an alarm is applyable to the recipient via an internal alarm system of the implantable component. Further, in an exemplary embodiment of this embodiment, the implantable component is configured to operate in at least two different operation modes, including the first mode detailed above in this paragraph (which has been referred to herein as a second mode elsewhere) and a second mode (which has been detailed herein in some instances as a first mode) that is a recipient-active mode of at least 6 hours in length where data is at least sometimes streamed from the implantable component to an external component, and an alarm is applyable to the recipient via an internal alarm system of the implantable component. In this embodiment, the first modes detailed outside of this paragraph correspond to the second modes detailed in this embodiment of this paragraph, and the second modes detailed outside of this paragraph correspond to the first mode of this embodiment of this paragraph.

Concomitant with the embodiments above, the implantable prosthesis can be an EKG or an EEG monitor.

Some embodiments include a system, which includes an implantable apparatus as detailed herein, and two external devices. A first of the two external devices can be a body worn external device that is a dedicated external device that is utilized with the implantable device. As detailed above, in at least some exemplary embodiments, the external device includes an arrangement to align the external inductance coil of the implantable inductance coils. A second of the two external devices can be a non-body worn device configured for use during the second mode. As detailed above, in an exemplary embodiment, this can be the pillow charger or the tunic charger, etc. such a system distinguishes from a system that is limited to only, for example, the components associated with FIG. 14 in FIG. 15 detailed above.

In an exemplary embodiment of the aforementioned system, the implantable apparatuses an EEG or an EKG monitor.

An exemplary embodiment includes an implantable EEG monitor or an EKG monitor or another type of monitor with no internal power supply that operates in two distinct operation modes. One of the modes is for day use where the recipient is conscious and/or active. The day mode can be such that the external component (BTE device, OTE device, etc.) is connected in close proximity to the implant. The day mode can be such that the external component provides power to the implant. The day mode can be such that the external component receives streamed data from the implant. The day mode can be such that the streamed data bandwidth is faster than the EEG recording bandwidth. The day mode can be such that the external component sends this data to another device that is not per se part of the prosthesis, such as a smartphone, or another remote device, and that data can be analyzed by the another device, and/or passed on to another location, such as a remote computer via the Internet or the like where the data is then analyzed. In an exemplary embodiment, the data can be analyzed at these remote components, and then, based on the analysis, and alarm or indication of the like can be provided to the external component, either directly or through the smart phone, etc., and the external component can then provide an alarm to the recipient or a health professional, or an emergency dispatch system to find and provide help to the recipient, etc. That is, the external component in some embodiments back simply as a pass-through device. In an exemplary embodiment, the time from which the external component passes the data to the remote device to the time where the external component receives data indicating that an alarm or the like should be provided to the recipient is within 3, 2.5, 2, 1.5, 1, 0.75, 0.5, 0.4, 0.3, 0.2, or 0.1 minutes. Additional details of such are described below.

The above said, the day mode can be such that the external component monitors this data for specific characteristics. That is, in this regard, the external component, such as, for example, the external component 1440 can be programmed to analyze the data and determine, based on the analysis, whether there is something about which the recipient should be warned, and then provide an alarm to the recipient. In an exemplary embodiment, this can be executed without the remote devices, such as the smart phone. Thus, in an exemplary embodiment, the day mode can be such that the external component provides alarms to the user.

Further with respect to this system, one of the modes of the first device having no battery therein can be a night mode where the recipient is resting or sleeping or otherwise passive. The night mode can be such that another external device different in type from the external device that is normally used with the prosthesis/the device used during the first mode/day mode, is used to provide power to the implant and/or provide data to/obtain data from the implant. In an exemplary embodiment, this device that is different from the device utilized during the day mode can be the pillow charger or the sheet charger, etc., as detailed above. The night mode can be such that another additional device is used to receive information from the implant (wireless Bluetooth, streaming via the inductance coils, etc.). The night mode can be such that a different communication method (one that does not rely on close proximity) is used to communicate with the external device.

In an exemplary embodiment, the implanted device is configured with a communication system that is different than the RF inductance communication system. In an exemplary embodiment, the implantable component can include a Wi-Fi or a Bluetooth communication system that can communicate with a component that is located away from the recipient. Some additional features enable such are described in more detail below. That said, the night mode can be such that the implantable device stores data, such as EEG data and/or EKG data. The night mode can be such that the implantable device analyses EEG data and/or EKG data to identify the occurrence of a specific event (which can warrant the issuance of an alarm or other indication to the recipient). The night mode can be such that if a specific event occurs, the EEG data is stored at a high resolution (higher than that associated with normal/non event occurrence recording). The night mode can be such that if an event occurs an alarm is provided to the user, where the alarm is provided through an implanted tissue stimulator attached to the implant (again, additional details of which are described below). The night mode can be such that the alarm/indication is provided to the user through communication with an external device as well.

As seen from the above, the night mode is a mode in which the implantable component undertakes actions that are more power intensive than that which is undertaken during the day mode, at least in some instances. In this regard, in an exemplary embodiment, utilizing the charging devices detailed herein, it is possible to provide more power to the implantable component relative to that which is the case if only the traditional dedicated external device, such as the device of FIG. 14, was utilized. In this regard, while the device of FIG. 14 may not necessarily be sufficient to power an implanted Bluetooth communication system and/or an implanted Wi-Fi communication system, or if such was sufficient in the short run, the end result on the battery with a power source of the external component would be such that the battery of the external component is quickly drained, while the other charging systems detailed herein can be sufficient to power such implanted systems. For example, whatever communication regime is utilized, communicating with a component that is located away from the skin of the recipient, as opposed to communicating with the headpiece 1478 of the embodiment of FIG. 14, will require much more power from the implanted component. Indeed, such can be the case because the charging systems are connected to household power sources as opposed to relying on batteries, or alternatively, are connected to larger batteries (e.g., backup power batteries, such as those used for computers and the like). Accordingly, during the night mode, more intensive power consuming actions can be undertaken by the implantable component relative to that which would otherwise be the case without the night mode. Indeed, this is somewhat counterintuitive in that typically, because implantable systems rely on the external component such as the component of FIG. 14 for power, and such component is not typically worn during the night, the functions of the implant are actually reduced, if not eliminated, while the recipient is sleeping.

Thus, in an exemplary embodiment of method 1800, the implanted medical device consumes at least G times as much power on a per hour basis during the second temporal period than during the first temporal period with respect to function not associated with internal power storage componentry. In an exemplary embedment, G is 1.1, 1.5, 2, 2.5, 3, 3.5, 4, 4.5, 5, 5.5, 6, 6.5, 7, 7.5, 8, 9 or 10 or more.

To be clear, in an exemplary embodiment, during the night mode, the implanted component can communicate on a continuous or at least a semi-continuous basis with a component that has an antenna that is at least 0.1, 0.25, 0.5, 0.75, 1, 1.25, 1.5, 1.75, 2, 2.5, 3, 3.5, 4, 4.5, 5, 5.5, 6, 6.5, 7, 8, 9, 10, 11, 12, 13, 14, or 15 meters away from the implanted antenna of the implanted device, providing that the art enable such. This as opposed to the embodiments where during the day mode, the implantable component, or more accurately, the implanted antenna, is communicating with an external antenna that is no more than 5, 4, 3, 2, 1.5, 1, 0.75, 0.5, or 0.25 centimeters away. Herein, the latter distances are deemed to be within the realm of close proximity to the implanted antenna.

Note also the ability of the implanted device to store data during the night mode. While this capability is not mutually exclusive with the day mode, this feature is something that again, may not be readily available on implants during the night mode of operation without the innovations detailed herein.

Of course, the ability to analyze data obtained from the sense electrodes is something that is power intensive, relative to merely recording such, at least in some exemplary embodiments. Corollary to this is the action of providing the alarm, which is also power intensive relative to not providing an alarm, at least where the alarm is provided from the implantable devices opposed to an outside device.

Thus, in an exemplary embodiment, at least some scenarios of use during the nighttime mode results in power consumption that is at least 1.5, 2, 3, 4, 5, 6, 7, 8, 9, 10 times or more than that which would otherwise be the case when utilizing only the traditional external component to power the implant, where the aforementioned power consumption can last for at least 10, 15, 20, 25, 30, 35, 40, 45, 50, 60, 70, 80, 90, 120, 150, 180, 210, 240, or 300 minutes or more.

An exemplary embodiment includes an implantable EEG monitor or another type of monitor with an internal power supply that operates in two distinct operation modes. One of the modes is for day use where the recipient is conscious and/or active. The day mode can be such that the implanted component operates autonomously without any external component, although in some embodiments, the implantable component can also operate in the day mode with an external component. Concomitant with the teachings detailed above, the day mode can be such that the implanted component receives power only from an implanted battery or other power source that is implanted in the recipient. In this exemplary embodiment, the implant monitors and/or stores data, such as EEG and/or EKG data, during the day mode of operation. Also, in at least some exemplary embodiments, during the day mode of operation, the implantable component can analyze the data, and can make a determination as to whether or not an alarm should be provided to the recipient based on the data. In an exemplary embodiment, the alarm is provided according to the teachings detailed herein utilizing componentry all of which is implanted in the recipient.

It is noted that the type and/or quality and/or quantity of the alarm can be based on the state of the implanted battery. For example, if the implanted battery is at a low level of charge, the alarm might be an alarm that utilizes lower power (e.g., the actuation of an actuator to provide a tactile sensation as opposed to the energizement of electrodes to provide an electrically based hearing percept), and vis-a-versa. Also, while in the day mode, this device can be configured so as to provide an alarm to the recipient depending on the state of the implant's memory. By way of example only and not by way of limitation, if the memory is getting full, the alarm or otherwise indication to the recipient can notify the recipient in some form or another that the recipient should in short order obtain an external device so that the data that is stored in the implanted memory can be uploaded to the external device.

Consistent with the teachings detailed above, in at least some exemplary embodiments, the implanted device can operate in a night mode where the user is unconscious or otherwise sleeping or otherwise is resting. In this exemplary embodiment, the implantable component can receive power for operation and/or to charge the battery from one of the external components other than the external component that is utilized in the traditional manner with the implant (e.g. the device of FIG. 14—to be clear, all totally implantable devices require an external device, if only to charge the implanted battery—the device of FIG. 14 provides such ability, and thus is a traditional external component utilized with a totally implantable component).

During the night mode, the implantable device can monitor the EEG signals and/or the EKG signals and/or can store the data. That said, in at least some exemplary embodiments, the implantable device can instead or also stream the data to a remote device according to any of the teachings detailed herein, where the data is analyzed. Still, there is utilitarian value with respect to enabling the implanted device to analyze the data, such as in the scenario where, for example, the communication system with the external device/remote device fails, if only with respect to data transmission (power can still be transferred in some scenarios, while in other scenarios, power can be halted as well).

During the night mode, as with the day mode, the implantable component can be configured to provide internal alarms to the recipient utilizing totally implantable devices. That said, in alternate embodiments, the implantable component can utilize the longer range (non-close proximity) mitigation systems to communicate data indicating that an alarm should be provided to the recipient, such as to communicate with a remote device that is configured to activate the alarm (e.g., flash lights, operate a siren, etc.), or a medical practitioner, or a medical dispatch group such as ambulance, etc.

At least some exemplary embodiments of the night mode of operation include the ability to stream data from the implantable component. In an exemplary embodiment, the data is streamed to an external component in close proximity to the implant (pillow charger, sheet charger, etc.). In an exemplary embodiment, the external component, such as the black box 930, which can contain memory and/or can be a personal computer or the like, can record that data and store that data. Of course, in at least some exemplary embodiments, as detailed above, while the data is being streamed, the external component can provide power to the implant so that the implant can stream the data outside of the implant. Again, in an exemplary embodiment, the utilization of the charging devices that are different than the traditional external component can enable the implant to operate at a power consumption level that is much higher than that which would be the case utilizing the traditional external component of the prostheses. In an exemplary embodiment, the external device, such as black box 930, can be configured to analyze data that is streamed and execute the enunciation of an alarm, either via a component on the black box such as a light, a noisemaker, etc., or via communication with a mother system, such as a household alarm system, where the black box 930 instructs the alarm system to create an alarm or to notify a medical practitioner, obtain an ambulance, etc.

As can be seen, there is utilitarian value with respect to the utilization of the implantable component having a power source. Such can enable continual operation of the implantable component when the external component is not in signal communication with the implantable component, such as, in a scenario where the recipient is taking a shower, getting dressed, getting his or her haircut, etc. Indeed, in some exemplary scenarios, the utilization of the implanted component with its own separate power source can have utilitarian value in other scenarios where the external component comes off of the recipient or otherwise ceases to be in signal communication with the implantable component, such as, by way of example only and not by way of limitation, where a person has a seizure, experiences a sudden deceleration, suffers from some form of event that causes the recipient to fall to the ground (cardiac arrest), etc., or even normal physical activity. Such can be very utilitarian with respect to recipients who need an EEG monitor relative to other people and a statistically significant population (e.g., those prone to epilepsy).

Indeed, some exemplary embodiments enable EEG and/or EKG monitoring for at least 1, 2, 3, 4, 5, 6, 7, 8, 9, 10, 11, 12, 13, 14, 15, 16, 17, 18, 19, or 20 hours or more without being recharged and/or without being in signal communication with an external device. Indeed, some embodiments enable EEG and/or EKG monitoring for the aforementioned period of times a totally implanted system.

Teachings detailed herein can be applicable to management or otherwise the monitoring of epilepsy prone peoples. In this regard, seizure events can be infrequent, with many months between events. Diagnosis requires at least one seizure to be captured. Many patients remain undiagnosed or incorrectly diagnosed due to lack of long term monitoring. Utilizing the teachings detailed herein, as can be seen, can provide EEG data capturing prior to and/or during a seizure. Accordingly, some exemplary methods include practicing the details herein respect to a method of treating and/or monitoring epilepsy.

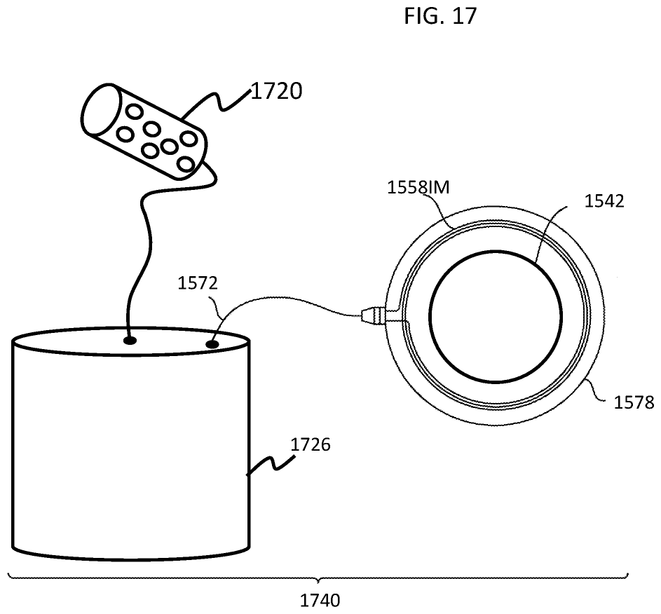

It is noted that while the embodiments detailed herein have focused on electrical detection/electrical monitoring/electrical analyses (ECE/EEG), other embodiments are related to detecting/monitoring, analyzing changes in the chemical composition of substances inside a body. By way of example only and not by way of limitation, FIG. 17 provides a schematic of an implantable component 1740 that is configured to monitor body fluid chemistry. In this regard, there is housing 1726 that includes a processor or the like that is program to analyze data via a signal from blood capture device 1720. The blood capture device 1720 is configured to capture blood and/or to analyze the blood to evaluate the chemistry thereof. By way of example only and not by way of limitation, the implantable component 1740 can be a blood glucose implant monitor that monitors blood directly or indirectly to determine its glucose level. The captured blood then is analyzed by a device 1726.

Note further that in an exemplary embodiment, the implantable component 1740 can be a new drug analyzer. By way of example only and not by way of limitation, the implantable component 1740 can be configured or otherwise programmed to analyze blood chemistry to evaluate the effects of a new drug.

The above said, it is noted that in at least some exemplary embodiments, an EEG system can be utilized to evaluate blood glucose levels and/or new drug efficacy. In this regard, there can be a scenario of use where there is a new drug introduction, and the evaluation regime of the new drug introduction includes brain monitoring, where the brain monitoring includes application of an EEG monitoring. At least some of the exemplary embodiments detailed herein provide enablement for continuous monitoring, and such can be very utilitarian for new drug evaluation.

It is briefly noted that a tertiary monitoring method through EEG analysis can detect hypoglycemia (low blood sugar levels). To maximize utilitarian value, the implantable component can be monitored continuously, and long term.

Traditionally, the problem associated with monitoring the above-noted phenomenon is that if the data is to be streamed in real-time or semi-real-time, an external component is required. Again, typically, the external component is an external component that is worn on the head. During sleep or a seizure though, this component would often be removed, or fall off. Accordingly, the teachings detailed herein can provide for the streaming and/or the recordation of the data in the complete absence of the traditional external component that is utilized with the implant.

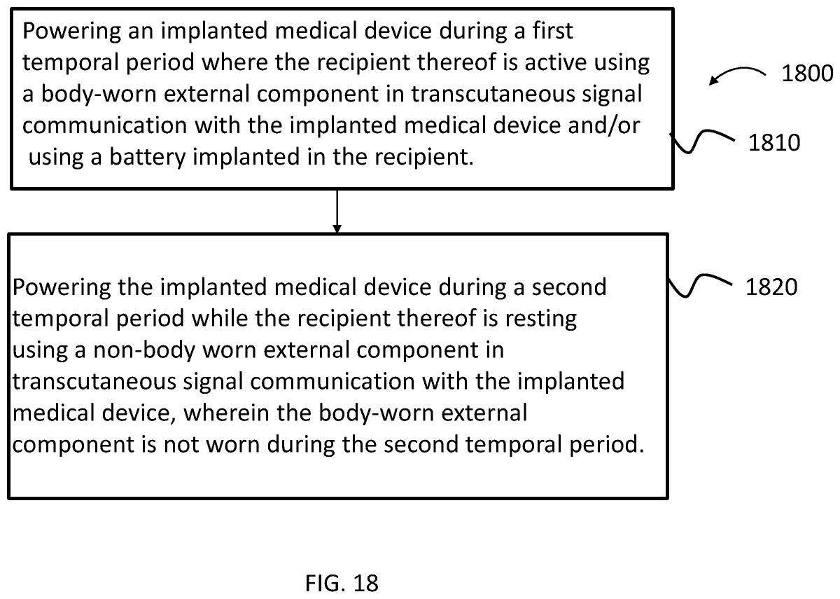

Embodiments include methods. FIG. 18 presents an exemplary algorithm for an exemplary method, method 1800, which includes method action 1810, which includes powering an implanted medical device (e.g., an EEG monitor) during a first temporal period where the recipient thereof is active (e.g., working, playing, functioning in a manner having body functionality elevated relative to that which would be the case when engaging in leisure sitting around/laying around and/or sleeping) using a body-worn external component in transcutaneous signal communication with the implanted medical device and/or using a battery implanted in the recipient. In an exemplary embodiment, the body worn external component is the traditional body worn external component that is utilized to power and/or communicate with the implantable component.

Method 1800 also includes method action 1820, which includes powering the implanted medical device during a second temporal period while the recipient thereof is resting using a non-body worn external component is in transcutaneous signal communication with the implanted medical device, wherein the body-worn external component is not worn during the second temporal period. In an exemplary embodiment, the second temporal period does not overlap the first temporal period. That said, in an alternate exemplary embodiment, the second temporal period overlaps the first temporal period. By way of example only and not by way of limitation, in a scenario where continuous monitoring or the like is deemed to be utilitarian, the recipient could jump into bed or the like with the body worn external component worn on the recipient's head, for example, and then, after a determination is automatically made that the non-body worn component has taken over at least some of the functionality of the body worn component, which determination can be made by the implant and/or by the external component and/or by the non-body worn external component, etc., or any other device that can make such a determination, the body worn component is removed or otherwise shut down. In an exemplary embodiment, the first temporal period corresponds to the temporal period associated with the day mode of operation detailed above, while the second temporal period corresponds to the temporal period associated with the night mode of operation detailed above.

In an exemplary variation of the above method, both the external device used during the first temporal period and the non-body worn external component used during the second temporal period are simultaneously used during a third temporal period after the first and second temporal periods. In an exemplary embodiment, the implantable component can be powered by both components simultaneously. In an exemplary embodiment, the implantable component can receive data or otherwise non-power signals from one of the components and receives power from the other component. Actually, the implantable component may receive both power and data from both components and/or receive power from both components, but in some embodiments, is configured to utilize only the nonpower signal from one component and the power signal from the other component. Further, in an exemplary variation, both the external device used during the first temporal period and the non-body worn external component are simultaneously detected as being in powering range and/or signal range (useful signal range) by the implanted medical device during a third temporal period after the first and second temporal periods, and the method further includes selectively receiving and/or using a power signal from one of the two devices to the exclusion of the other of the two devices.

Also, in an exemplary embodiment of the aforementioned method, both the external device used during the first temporal period and the non-body worn external component are simultaneously used during a third temporal period after the first and second temporal periods to execute at least some of the respective actions that have occurred during the first temporal period and the second temporal period.

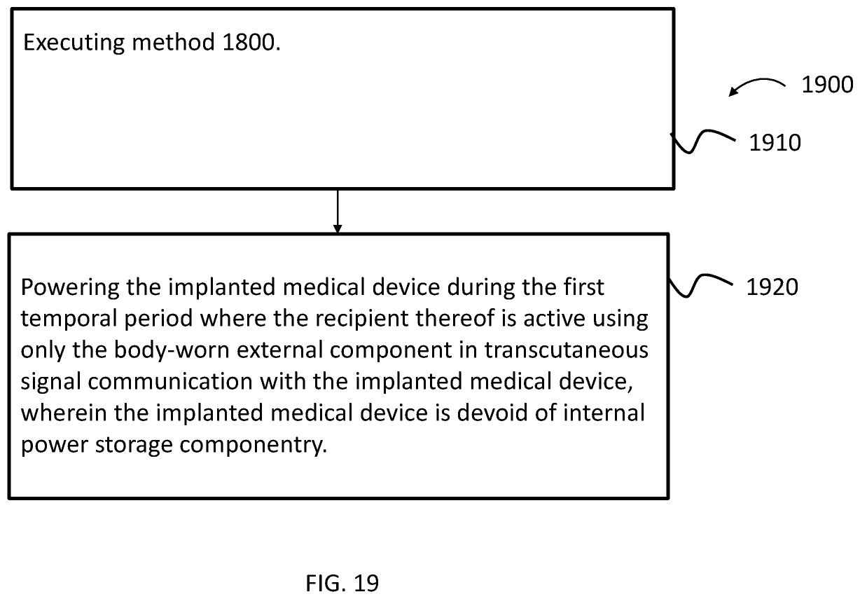

FIG. 19 presents an exemplary algorithm for an exemplary method, method 1900, which includes method action 1910, which includes executing method 1800. Method 1900 also includes method action 1920 which includes the action of powering the implanted medical device during the first temporal period where the recipient thereof is active using only the body-worn external component in transcutaneous signal communication with the implanted medical device, wherein the implanted medical device is devoid of internal power storage componentry.

In a variation of method 1900, there is the action of powering the implanted medical device for an uninterrupted period of at least H hours during the first temporal period where the recipient thereof is active using only the battery implanted in the recipient, where H can be 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 13, 14, 15, 16, 17, or 18 or more hours.

In an alternate embodiment of method 1800, there is the action of streaming data from the implanted medical device to the body worn external component during the first temporal period and storing data in the implanted medical device during the second temporal period. In an exemplary embodiment, there is no streaming of data during the second temporal period and/or there is no storage of data in the implantable component during the first temporal period. In another exemplary embodiment, there is also streaming of data during the second temporal period and there is also storage of data in the implantable component during the first temporal period. Thus, in an exemplary embodiment of the aforementioned method, there the action of streaming data from the implanted medical device to the body worn external component during at least a portion of the first temporal period and/or storing data in the implanted medical device during at least a portion of the first temporal period. Further, there is the action of streaming data from the implanted medical device to the non-body worn external component during at least a portion of the second temporal period and/or storing data in the implanted medical device during at least a portion of the second temporal period.

An exemplary method also includes, during first temporal period, automatically providing an alarm both internally and externally, and during the second temporal period, only providing an alarm internally/not providing any alarm externally.

Concomitant with the teachings detailed above, in an exemplary embodiment, the non-body worn external component is an accoutrement of a bed. (Pillow, sheet, etc.)

As noted above, embodiments include implantable components that are configured to provide indications, such as alarms, to a recipient thereof, in complete isolation/completely without any external component. In this regard, in an exemplary embodiment, there is an apparatus, comprising an implantable component of an implantable prosthesis, the implantable component configured to autonomously provide a perceptible meaningful indication related to an operation of the implantable prosthesis to a recipient thereof totally via implanted componentry.

Again, as noted above, there can be embodiments that include a third mode, and/or a fourth mode can be an alarm mode, where an alarm can be raised while in one of the other modes. The user would then place an external component on the head to provide power or to stream data out from the implant. The following provides an example of a system that can enable such. It is noted that in some embodiments, the third mode can be a mode in which the implantable component provides a status to the recipient, and the fourth mode can be an alarm mode. Both of these modes are indication modes. The modes of the prosthesis can be such where the implantable component operates differently depending on a given mode. By way of example, in a mode where the prosthesis is providing the alarm, the prosthesis can also go into a functional mode that maximizes some functionalities over others. By way of example only and not by way of limitation, in some embodiments, the functionality of the prosthesis with respect to streaming data might be suspended because the determination has already been made that there is an occurrence of an issue, such as a functional error or a physiological event, etc. The prosthesis can instead utilize the assets thereof to maximize other more important features, such as potentially recording the data at a higher resolution. Conversely, during the mode that is a status mode, the functionality of the prosthesis might be the exact same as that of one of the other modes.

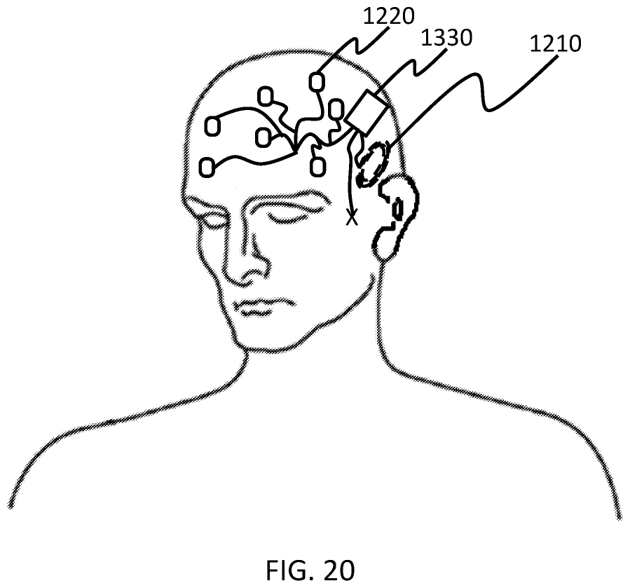

FIG. 20 presents an exemplary embodiment of a modified version of the embodiment of FIG. 13 detailed above. Here, an extra electrode is provided at the location represented by the “X.” This extra electrode is an extra-cochlear electrode positioned in proximity to the cochlea of the recipient such that when this electrode is energized, a hearing percept sensation occurs. This is somewhat analogous to how the cochlear implant of FIG. 1 operates, except that the electrodes are completely outside the cochlea. The purpose of this electrode and this arrangement is not to evoke a hearing percepts corresponding to speech or the like, but instead to simply evoke a hearing percept that the recipient perceives in a manner that the recipient will recognized as an indication or an alarm from the implant. The hearing percept can be a beep, a static sound, or any sound that can be created by such an arrangement. Indeed, in an exemplary embodiment, the hearing percept is supposed to be annoying/one that gets the recipient's attention. The electrode can be energized and the energized via a pattern that is predetermined. By executing a method where the recipient is exposed to this pattern in a clinical setting or any other nonemergency/non-event scenario where the recipient learns what the indication “sounds like,” the recipient can correlate a given hearing percept and/or a given pattern of the hearing percept to an alarm or otherwise an indication.

In an exemplary scenario of use, upon the implantable component providing the indication to the recipient by energizing the electrode, the recipient, who has been previously instructed that upon such indication the recipient should obtain his or her external component that is traditionally used with a device, the recipient puts on the external device/body worn device. In an exemplary embodiment, this can stop the indication. That said, in an exemplary embodiment, the implantable component can include a so-called failsafe system that enables the recipient to stop the indication, such as by way of example only and not by way of limitation, placing some form of metallic component adjacent the housing 1330 and/or taping the metal housing a certain number of times in quick succession, etc.

The point is that in an exemplary embodiment, the implantable component is configured not only to monitor the recipient's body, but also to provide an indication to the recipient. In an exemplary embodiment, the housing 1330 can include a cochlear implant speech processor and/or sound processor that is configured to output stimulation signals that can evoke a hearing percept. In this regard, in an exemplary embodiment, the processor located in the housing 1330 can be a rather “low-tech”/“unsophisticated” processor, as the processor is not specifically designed to operate as a cochlear implant so that the recipient can understand captured speech or the like. That said, in some embodiments, even with these low-tech solutions, the hearing percept can be words or something resembling words. By way of example only and not by way of limitation, an electrically stimulated hearing percept corresponding to “seizure warning” or the like could be provided by the system, potentially with only extra-cochlear electrodes.

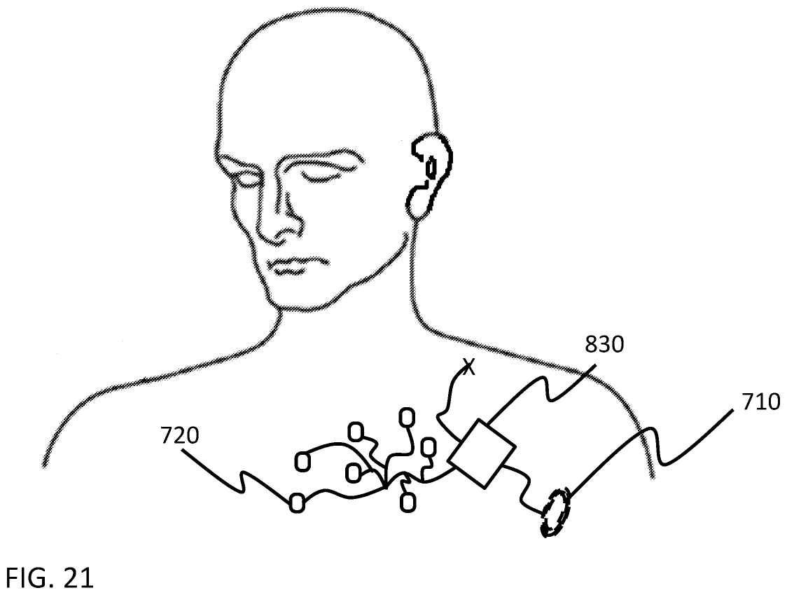

FIG. 21 provides an exemplary EKG monitoring system where another electrode represented by the “X” is located away from the heart. Instead of evoking a hearing percept, the electrode can induce a sensation of pain or the like at a location away from vital tissue. By way of example only and not by way of limitation, the electrode can be located at the shoulder region. Alternatively, instead of pain, a tingling sensation can be potentially induced. The pain/tingling, etc., can be presented in an on/off manner so as to represent some form of indication or warning, providing that the predetermined pattern is known to the recipient.

It is briefly noted that in at least some exemplary embodiments, there may not necessarily be an extra electrode or separate electrode that provides the stimulation. In an exemplary embodiment, it is possible that one or more of the read electrodes is utilized as a stimulation electrode. It is also briefly noted that while the embodiments detailed above have been described in terms of a single electrode, it is noted that at least two electrodes can be utilized, one as a source and one as a sink. It is noted that in electrode can be positioned on the implanted housing and/or the housing can be utilized as one electrode in at least some exemplary embodiments.

While the embodiments detailed above have focused on the utilization of an electric signal applied to tissue to evoke the indication, in an alternate embodiment, another type of tissue stimulator can be utilized. By way of example only and not by way of limitation, in an exemplary embodiment, a vibratory device can be implanted into the recipient with the implanted device. By way of example only and not by way of limitation, in an exemplary embodiment, a bone conduction vibrator can be implanted at the locations of the “X” (it is noted that any of the stimulators detailed herein can be implanted anywhere providing that such can enable the teachings detailed herein and providing that such does not threaten the life of the recipient—the depictions of the locations of the tissue stimulators are presented for exemplary purposes only). Alternatively, and/or in addition to this, a middle ear actuator can be implanted as the tissue stimulator. In some embodiments, these components evoke a hearing percept, while in other embodiments, there is no hearing percept that is evoked, and instead, the sensation of the vibrations and/or movement is what is utilized to provide the indication of the recipient. Indeed, in an exemplary embodiment, the aforementioned bone conduction vibrator is not so much for bone conduction as it is simply to provide a tactile sensation beneath the skin. Indeed, in an exemplary embodiment, such as for example with respect to the middle ear actuator, the actuation thereof can potentially simply pinch the skin or otherwise provide some potentially irritating sensation. This sensation by itself can provide the indication/warning to the recipient, while in other embodiments, a pattern of irritation can be implemented.

The vibrations can be controlled such that the tactile sensation is presented in a pattern, which pattern is pre-known to the recipient and thus indicative of an indication. Still, in some exemplary embodiments, a bone-conduction hearing percept can be evoked. As with the electrical stimulation, the bone conduction hearing percept need not necessarily be speech, but instead can be more general sounds. That said, in some embodiments, speech can be evoked. As with the embodiments detailed above, in some embodiments, a bone-conduction sound processor can be implanted in the recipient, albeit perhaps a low-tech device, that can control the bone conduction vibrator to reproduce a speech sensation to provide the indication. Such can be also the case with respect to a middle ear actuator.

It is briefly noted that while some embodiments utilize an extra electrode as shown, other embodiments could potentially use one or more of the read electrodes to provide electrical stimulation to the tissue of the recipient, providing that such is safe.

Note also that the mechanical transducer utilized in some embodiments to provide the indication need not necessarily have anything to do with a hearing prosthesis. By way of example only and not by way of limitation, an implanted vibrator akin to that which operates when a cell phone is in silent mode can be utilized. By way of example only and not by way of limitation, an unbalanced mass can be attached to a mechanical motor. The motor is usually in the off state, but upon the implanted component determining that an indication or a warning should be provided to the recipient, the motor is energized, and because the masses unbalanced, the housing in which the motor and the mass is located “shakes”, thus generating vibrations or otherwise providing the tactile sensation to the recipient.

It is specifically noted that in at least some exemplary embodiments, the implantable apparatus is not a hearing prosthesis as that would be understood by the person of ordinary skill in the art. In this regard, simply because the device evokes a hearing percept does not mean that it is a hearing prosthesis. As used herein, the phrase hearing prosthesis means that the device is configured to capture sound and evoke a hearing percept based on the captured sound. The teachings detailed herein that utilize a hearing percept to provide an indication to the recipient specifically do not require captured sound. In this regard, the implantable component is preprogrammed and/or preconfigured to evoke only a limited number of hearing percepts irrespective of the environment.

That said, in at least some exemplary embodiments, the teachings detailed herein can be combined with a hearing prosthesis or otherwise are even limited to a hearing prosthesis. In this regard, in an exemplary embodiment, the implantable component is an implantable component of a hearing prosthesis that includes a tissue stimulator that provides the indication.

Conversely, in an exemplary embodiment, the implantable component is an implantable component of a non-hearing prosthesis that includes a tissue stimulator that provides the indication.

In an exemplary embodiment, the implantable component includes a tissue stimulator that provides the indication. The tissue stimulator can be part of an apparats that provides additional functionality beyond (i) stimulating tissue to provide the indication (e.g., the system can be an EEG monitor, an EKG monitor, a body fluid monitor, a drug efficacy monitor, etc.) and (ii) if the implantable component is configured to provide functionality of a hearing prosthesis, stimulating tissue to provide a hearing percept based on external stimulation. External stimulation includes sound captured by sound capture apparatus, streamed audio to the hearing prosthesis, etc.

In an exemplary embodiment, the implantable component is part of a body monitoring device configured to monitor aspects of a recipient's body, wherein the implantable component is configured to evaluate the monitored aspects and determine if an aspect is outside of a given parameter, and upon such determination, provide the indication to the recipient, wherein the indication is an indication that an aspect is outside of a given parameter. Again, as detailed above, in an exemplary embodiment, the EEG monitor and monitor signals for a potential seizure or the like. The implantable component can analyze the signals in real time or near real time, and if the signals are indicative of a potential seizure, alert the recipient by providing the indication, which indication would be a warning that a seizure could be imminent.

In an exemplary embodiment, the indication is at least one of indicative of a status of the implantable component or indicative of a set feature of the implantable component. With respect to the former, such can be a battery status, and on or off status, etc. With respect to the latter, this can correspond to a given setting of the implantable component (aggressive monitoring for any variation in the signals, light monitoring for only extreme variation in the signals, etc.). Indeed, in an exemplary embodiment, the recipient may undergo external stimulation that will cause the signals read by the implantable component to vary. Because this stimulation is known to the recipient and expected to cause a variation, the recipient could adjust the implantable system to account for such. The warnings of the alarms of the indications would be periodically provided to the recipient so that the recipient understands that the setting of the system has changed or the like, thus reminding the recipient of the status of the implant.

In an exemplary embodiment, the implantable component is configured to stimulate tissue utilizing Morse code. In an exemplary embodiment, the implantable component is configured to stimulate to utilizing the 5×5 matrix of the alphabet without the letter Q (1 and 1 is A, 5 and 5 is Z, 2 and 1 is F, 2 and 2 is G). In an exemplary embodiment there is an exemplary scenario of use where the system provides a stimulation and the recipient writes down the code so that the recipient can understand what the system is “telling” him or her.

While the embodiments detailed above have focused on utilizing a low-tech sound processor or the like, it is noted that in at least some exemplary embodiments, even for systems that are not hearing prostheses, a full-fledged speech processor can be implemented into the implantable component. The system does not utilize the full capabilities of the speech processor. Because the speech processors are readily available, it can be economically utilitarian to use such even though such provides far more capability than that which will be needed.

Indeed, in an exemplary embodiment, some of the monitors are implemented with a hearing prosthesis, but the hearing prostheses is not in an active state. The hearing prosthesis can be activated if such is utilitarian at a later date.

It is further noted that any disclosure of a device and/or system detailed herein also corresponds to a disclosure of otherwise providing that device and/or system and/or utilizing that device and/or system.

It is also noted that any disclosure herein of any process of manufacturing other providing a device corresponds to a disclosure of a device and/or system that results there from. Is also noted that any disclosure herein of any device and/or system corresponds to a disclosure of a method of producing or otherwise providing or otherwise making such.

Any embodiment or any feature disclosed herein can be combined with any one or more or other embodiments and/or other features disclosed herein, unless explicitly indicated and/or unless the art does not enable such. Any embodiment or any feature disclosed herein can be explicitly excluded from use with any one or more other embodiments and/or other features disclosed herein, unless explicitly indicated that such is combined and/or unless the art does not enable such exclusion.

While various embodiments of the present invention have been described above, it should be understood that they have been presented by way of example only, and not limitation. It will be apparent to persons skilled in the relevant art that various changes in form and detail can be made therein without departing from the spirit and scope of the invention.

1. An apparatus, comprising:
an implantable component of an implantable prosthesis, the implantable component configured to autonomously provide a perceptible meaningful indication related to an operation of the implantable prosthesis to a recipient thereof totally via implanted componentry.

2. The apparatus of claim 1, wherein the implantable component is an implantable component of a hearing prosthesis that includes a tissue stimulator that provides the indication.

3. The apparatus of claim 1, wherein the implantable component is an implantable component of a non-hearing prosthesis that includes a tissue stimulator that provides the indication.

4. (canceled)

5. The apparatus of claim 1, wherein the implantable component is part of a body monitoring device configured to monitor aspects of a recipient's body, wherein the implantable component is configured to evaluate the monitored aspects and determine if an aspect is outside of a given parameter, and upon such determination, provide the indication to the recipient, wherein the indication is an indication that an aspect is outside of a given parameter.

6. (canceled)

7. The apparatus of claim 1, wherein the implantable component is part of an EEG monitor implanted in the recipient.

8. The apparatus of claim 1, wherein the implantable component is part of a drug delivery system and/or a drug monitoring system implanted in the recipient.

9. (canceled)

10. The apparatus of claim 1, wherein the implantable component is configured to monitor a recipient for an impending seizure and provide the perceptible meaningful indication upon a determination by the implantable component of an impending seizure.

11. A method, comprising:
powering an implanted medical device during a first temporal period where the recipient thereof is active using a body-worn external component in transcutaneous signal communication with the implanted medical device and/or using a battery implanted in the recipient; and
powering the implanted medical device during a second temporal period while the recipient thereof is resting using a non-body worn external component in transcutaneous signal communication with the implanted medical device, wherein the body-worn external component is not worn during the second temporal period.

12-13. (canceled)

14. The method of claim 11, further comprising:
powering the implanted medical device for an uninterrupted period of at least four hours during the first temporal period where the recipient thereof is active using only the battery implanted in the recipient.

15-16. (canceled)

17. The method of claim 11, further comprising:
during first temporal period, automatically providing an alarm both internally to the recipient and externally to the recipient; and
during the second temporal period, only providing an alarm internally to the recipient.

18. The method of claim 11, wherein:
the non-body worn external component is an accoutrement of a bed.

19. The method of claim 11, wherein:
the implanted medical device is a body sensor that records data indicative of a state of a body, and
the implanted medical device records the data at a higher resolution during the second temporal period than during the first temporal period.

20-21. (canceled)

22. The method of claim 11, wherein:
both the external device used during the first temporal period and the non-body worn external component are simultaneously detected as being in powering range by the implanted medical device during a third temporal period after the first and second temporal periods, and selectively receiving and using a power signal from one of the two devices.

23. The method of claim 11, wherein:
both the external device used during the first temporal period and the non-body worn external component are simultaneously used during a third temporal period after the first and second temporal periods to execute at least some of the respective actions that have occurred during the first temporal period and the second temporal period.

24. An apparatus, comprising:
an implantable component of an implantable prosthesis, the implantable component configured to operate in at least one operation mode, including a first mode is a recipient-passive mode of at least 4 hours length where the recipient sleeps, where the implantable component is powered for functional operation primarily from an external device not magnetically coupled to the recipient, where data is at least sometimes stored internally to the implantable component, and an alarm is applyable to the recipient via an internal alarm system of the implantable component.

25. The apparatus of claim 24, wherein:
the implantable component is configured to operate in at least two different operation modes, including the first mode and a second mode that is a recipient-active mode of at least 6 hours in length where data is at least sometimes streamed from the implantable component to an external component, and an alarm is applyable to the recipient via an internal alarm system of the implantable component.

26. The apparatus of claim 25, wherein:
the second mode is such that an alarm is only applied internally.

27. The apparatus of claim 25, wherein:
the second mode is such that the data is always streamed from the implantable component to the external component.

28. (canceled)

29. The apparatus of claim 24, wherein:
the first mode is such that the data is at least sometimes streamed from the implantable component to an external component.

30-33. (canceled)

34. The apparatus of claim 25, wherein:
the first mode is such that the data is at least sometimes streamed from the implantable component to an external component.
